(19) United States
(12) Patent Application Publication
(10) Pub. No.: US 2024/0416143 A1
(43) Pub. Date: Dec. 19, 2024

(54) PHOTONIC ANTIMICROBIAL WOUND SURFACE DRESSING

(71) Applicant: The General Hospital Corporation, Boston, MA (US)

(72) Inventors: Jeffrey A. Gelfand, Cambridge, MA (US); Richard Rox Anderson, Boston, MA (US); Laisa Bonafim Negri, Boston, MA (US); William A. Farinelli, Danvers, MA (US); Sandeep Korupolu, Boston, MA (US); Joshua Tam, Boston, MA (US)

(73) Assignee: The General Hospital Corporation, Boston, MA (US)

(21) Appl. No.: 18/702,947

(22) PCT Filed: Oct. 21, 2022
(86) PCT No.: PCT/US2022/078479

§ 371 Date: Apr. 19, 2024

Related U.S. Application Data
(60) Provisional application No. 63/270,249, filed on Oct. 21, 2021.

(51) Int. Cl. A61N 5/06; A61M 3/02; A61N 5/00

(52) U.S. Cl. CPC A61N 5/062; A61M 3/0262; A61N 5/0624; A61M 2202/0208; A61N 2005/005; A61N 2005/0626; A61N 2005/0645; A61N 2005/0652; A61N 2005/0663

(57) ABSTRACT

Devices and methods for improving the infection-healing behavior of a biological wound are disclosed. The device can comprise: a matrix; a light-emitting layer comprising a plurality of light sources; a controller in electrical communication with the light sources, the controller being configured to execute a program stored in the controller to activate the light sources to irradiate the wound or a region adjacent the wound with light for a period of time when the device is placed over the wound; and an antimicrobial adjuvant present as part of the device in an amount effective to synergistically potentiate antimicrobial activity of the light with respect to microbes on or adjacent the wound. The wound may include microbes in a biofilm. In one embodiment, the antimicrobial adjuvant comprises a substituted naphthalene, such as menadione. In one embodiment, the antimicrobial adjuvant comprises near infrared radiation.

### CROSS-REFERENCE TO RELATED APPLICATIONS

This application is based on, claims benefit of, and claims priority to U.S. Application No. 63/270,249 filed on Oct. 21, 2021, which is hereby incorporated by reference herein in its entirety for all purposes.

### STATEMENT REGARDING FEDERALLY SPONSORED RESEARCH

This invention was made with government support under one or more of Department of Defense agreements #2019A 00456, fund #235656, and grant number HU00011920056. The government has certain rights in the invention.

### BACKGROUND OF THE INVENTION

### 1. Field of the Invention

This invention relates to methods and devices for improving healing of infection as well as healing behavior of a biological wound, kits for improving healing of infection as well as healing behavior of a biological wound, and methods for killing or inhibiting growth of microbes in a biofilm such as bacteria in a biofilm on or adjacent a biological wound.

### 2. Description of the Related Art

Many types of large open wounds, such, as those associated with tissue grafts, burns, abrasions, ulcers and the like, require careful treatment to facilitate their closure and healing. A number of factors have been identified that promote healing of such wounds. These factors include, e.g., a sterile environment to avoid infection, an adequate blood supply to the wound region, sufficient oxygen and hydration, proper drainage, etc. Many types of wound dressings have been developed and studied for promoting healing of wounds.

In a study of wound infections in which antibiotics were antibiotic-susceptibility matched, treatment-induced resistance often resulted from rapid reinfection with a different strain, resistant to that antibiotic. The more prolonged and chronic the treatment, the more likely the development of that resistance. Complicating wound treatment is the fact that wound infections rapidly develop into biofilm infections, in which bacteria are encased in a matrix of bacterial carbohydrates, bacterial and host DNA, bacterial and host proteins, all further rendering biofilm-resident bacteria dramatically less sensitive to antibiotics.

Accordingly, there is a need to provide improved wound dressing devices and methods that provide one or more conditions conducive to healing of a biological wound.

### SUMMARY OF THE INVENTION

The present invention meets the foregoing needs for improved healing of infection as well as wound dressing devices and methods that provide one or more conditions conducive to healing of a biological wound.

In one aspect, the disclosure provides a device for improving healing behavior of a biological wound. The device comprises: a matrix; a light-emitting layer comprising a plurality of light sources; a controller in electrical communication with the light sources, the controller being configured to execute a program stored in the controller to activate the light sources to irradiate the wound or a region adjacent the wound with light for a period of time when the device is placed over the wound; and an antimicrobial adjuvant present as part of the device in an amount effective to synergistically potentiate antimicrobial activity of the light with respect to microbes on or adjacent the wound.

In one embodiment, the controller is configured to execute the program stored in the controller to activate the light source to irradiate the wound or a region adjacent the wound with light for the period of time such that the light is provided at a radiant exposure of at least 50 J/cm² during the period of time.

In one embodiment, the light has a wavelength that ranges from 380 nanometers to 700 nanometers. In one embodiment, the light has a wavelength that ranges from 380 nanometers to 410 nanometers.

In one embodiment, the plurality of light sources comprise an array of light emitting diodes in electrical communication with the controller, the controller being configured to execute a program stored in the controller to: (i) activate a first group of the light emitting diodes to irradiate the wound or the region adjacent the wound with light at a first range of wavelengths, and (ii) activate a second group of the light emitting diodes to irradiate the wound or the region adjacent the wound with light at a second range of wavelengths, the first range of wavelengths being different than the second range of wavelengths, and (iii) activate a third group of the light emitting diodes to irradiate the wound or the region adjacent the wound with light at a third range of wavelengths, the first range of wavelengths being different than the second range of wavelengths. In one embodiment, the first range of wavelengths ranges from 700 nanometers to 2000 nanometers, the second range of wavelengths ranges from 380 nanometers to 700 nanometers, and the third range of wavelengths ranges from 1000 nanometers to 1400 nanometers.

In one embodiment, the plurality of light sources comprise an array of light emitting diodes, and the light-emitting layer comprises at least one lens for focusing or dispersing light from at least a portion of the light emitting diodes.

In one embodiment, a translucent film positioned between the matrix and the light-emitting layer, wherein the film connects the matrix and the light-emitting layer.

In one embodiment, an antimicrobial adjuvant is present as part of the device in an amount effective to synergistically potentiate antimicrobial activity of the light with respect to microbes on or adjacent the wound.

In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix.

In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from minocycline, doxycycline, demeclocycline, and mixtures thereof.

In one embodiment, the matrix includes an associated bioactive agent selected from the group consisting of glycopeptide antibiotics.

In one embodiment, the controller is configured to execute the program stored in the controller to operate the light source using a plurality of cycles in which each cycle includes a first period of time in which the light source is on and a second period of time in which the light source is off. In one embodiment, the controller is configured to execute the program stored in the controller to operate the light sources using a plurality of cycles in which each cycle includes a first period of time in which a first group of the light sources is on and other groups of the light sources are off and a second period of time in which a second group of the light sources is on and other groups of the light sources are off. The first period of time can have a different length of time than the second period of time. The first period of time can have a same length of time as the second period of time.

The device can further comprise an oxygen sensor for sensing oxygen on or adjacent the wound, the oxygen sensor being in electrical communication with the controller. The device can further comprise a temperature sensor for sensing temperature on or adjacent the wound, the temperature sensor being in electrical communication with the controller. In one embodiment, the controller is configured to execute the program stored in the controller to turn off the light sources when the temperature sensor senses that the temperature has risen to a threshold temperature.

In one embodiment, the matrix has a surface including a channel, the surface facing the wound when the device is placed over the wound. The device can further comprise a source of irrigation fluid in fluid communication with an inlet of the channel. In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

In one embodiment, the matrix has a surface including a plurality of channels, the surface facing the wound when the device is placed over the wound. In one embodiment, a source of irrigation fluid is in fluid communication with an inlet of the channels. The device can further comprise a waste collector in fluid communication with an outlet of the channels.

In one embodiment, the matrix is formed from a plurality of matrix components, each matrix component having a first section and a second section, the first section of one matrix component being dimensioned to matingly engage the second section of an adjacent matrix component to form all or a part of the matrix.

In one embodiment, a coolant fluid is in contact with the matrix or the light-emitting layer. In one embodiment, the matrix includes a cavity for receiving the coolant fluid. In one embodiment, the device further comprises a pump for recirculating the coolant fluid, the pump being in fluid communication with the cavity, the pump being in electrical communication with the controller which activates and deactivates the pump. In one embodiment, the device further comprises a container including a coolant fluid, wherein the container is in contact with the matrix or the light-emitting layer. In one embodiment, the coolant fluid is selected from biocompatible fluids. In one embodiment, the coolant fluid is a dielectric fluid.

The light emitting layer can comprise LEDs with one or more wavelengths for different purposes to be illuminated either sequentially or simultaneously. The range of wavelengths transmitted by the LEDs can range from 380-1300 nm, from 400-470 nm; from 600-700 nm, from 700-800 nm, from 800-1300 nm, comprising blue light, green light (500-600 nm), red light (600-700 nm), and near IR. The controller can be programmed to enable duty cycles ranging from 0% to 90% “on”; the matrix (e.g., bandage) layer can be relatively transparent enabling light transmission and either capable or not of being complexed with adjuvant antimicrobial substances. Wavelengths with or without additional antimicrobial substances can be tailored to attack either polymicrobial infections, gram-positive bacterial infections, gram-negative bacterial infections, anaerobic or aerobic infections, mycobacterial and fungal or mold infections.

In another aspect, the disclosure provides a device for improving healing behavior of a biological wound. The device comprises: a matrix dimensioned to cover the wound, the matrix having a surface including a channel, the surface facing the wound when the device is placed over the wound; a light-emitting layer comprising a plurality of light sources; a controller in electrical communication with the light sources, the controller being configured to execute a program stored in the controller to activate the light sources to irradiate the wound or a region adjacent the wound with light for a period of time when the device is placed over the wound; and a source of irrigation fluid in fluid communication with an inlet of the channel.

In one embodiment, the controller is configured to execute the program stored in the controller to activate the light source to irradiate the wound or a region adjacent the wound with light for the period of time such that the light is provided at a radiant exposure of at least 50 J/cm² during the period of time.

In one embodiment, the light has a wavelength that ranges from 380 nanometers to 700 nanometers. In one embodiment, the light has a wavelength that ranges from 380 nanometers to 410 nanometers.

In one embodiment, the plurality of light sources comprise an array of light emitting diodes in electrical communication with the controller, the controller being configured to execute a program stored in the controller to: (i) activate a first group of the light emitting diodes to irradiate the wound or the region adjacent the wound with light at a first range of wavelengths, and (ii) activate a second group of the light emitting diodes to irradiate the wound or the region adjacent the wound with light at a second range of wavelengths, the first range of wavelengths being different than the second range of wavelengths, wherein the light at the first range of wavelengths is the antimicrobial adjuvant.

In one embodiment, the plurality of light sources comprise an array of light emitting diodes in electrical communication with the controller, the controller being configured to execute a program stored in the controller to: (i) activate a first group of the light emitting diodes to irradiate the wound or the region adjacent the wound with light at a first range of wavelengths, and (ii) activate a second group of the light emitting diodes to irradiate the wound or the region adjacent the wound with light at a second range of wavelengths, the first range of wavelengths being different than the second range of wavelengths. In one embodiment, the first range of wavelengths ranges from 600 nanometers to 2000 nanometers, and the second range of wavelengths ranges from 380 nanometers to 700 nanometers. In one embodiment, the controller is configured to execute the program stored in the controller to: (i) activate the first group of the light emitting diodes before activating the second group of the light emitting diodes.

In one embodiment, the plurality of light sources comprise an array of light emitting diodes in electrical communication with the controller, the controller being configured to execute a program stored in the controller to: (i) activate a first group of the light emitting diodes to irradiate the wound or the region adjacent the wound with light at a first range of wavelengths, and (ii) activate a second group of the light emitting diodes to irradiate the wound or the region adjacent the wound with light at a second range of wavelengths, the first range of wavelengths being different than the second range of wavelengths, and (iii) activate a third group of the light emitting diodes to irradiate the wound or the region adjacent the wound with light at a third range of wavelengths, the first range of wavelengths being different than the second range of wavelengths. In one embodiment, the first range of wavelengths ranges from 700 nanometers to 2000 nanometers, the second range of wavelengths ranges from 380 nanometers to 700 nanometers, and the third range of wavelengths ranges from 1200 nanometers to 1400 nanometers.

In one embodiment, the plurality of light sources comprise an array of light emitting diodes, and the light-emitting layer comprises at least one lens for focusing or dispersing light from at least a portion of the light emitting diodes.

In one embodiment, a translucent film positioned between the matrix and the light-emitting layer, wherein the film connects the matrix and the light-emitting layer.

In one embodiment, an antimicrobial adjuvant is present as part of the device in an amount effective to synergistically potentiate antimicrobial activity of the light with respect to microbes on or adjacent the wound.

In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix.

In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from minocycline, doxycycline, demeclocycline, and mixtures thereof.

In one embodiment, the matrix includes an associated bioactive agent selected from the group consisting of glycopeptide antibiotics.

In one embodiment, the controller is configured to execute the program stored in the controller to operate the light source using a plurality of cycles in which each cycle includes a first period of time in which the light source is on and a second period of time in which the light source is off. In one embodiment, the controller is configured to execute the program stored in the controller to operate the light sources using a plurality of cycles in which each cycle includes a first period of time in which a first group of the light sources is on and other groups of the light sources are off and a second period of time in which a second group of the light sources is on and other groups of the light sources are off. The first period of time can have a different length of time than the second period of time. The first period of time can have a same length of time as the second period of time.

The device can further comprise an oxygen sensor for sensing oxygen on or adjacent the wound, the oxygen sensor being in electrical communication with the controller. The device can further comprise a temperature sensor for sensing temperature on or adjacent the wound, the temperature sensor being in electrical communication with the controller. In one embodiment, the controller is configured to execute the program stored in the controller to turn off the light sources when the temperature sensor senses that the temperature has risen to a threshold temperature.

In one embodiment, the matrix has a surface including a channel, the surface facing the wound when the device is placed over the wound. The device can further comprise a source of irrigation fluid in fluid communication with an inlet of the channel. In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

In one embodiment, the matrix has a surface including a plurality of channels, the surface facing the wound when the device is placed over the wound. In one embodiment, a source of irrigation fluid is in fluid communication with an inlet of the channels. The device can further comprise a waste collector in fluid communication with an outlet of the channels.

In one embodiment, the matrix is formed from a plurality of matrix components, each matrix component having a first section and a second section, the first section of one matrix component being dimensioned to matingly engage the second section of an adjacent matrix component to form all or a part of the matrix.

In one embodiment, a coolant fluid is in contact with the matrix or the light-emitting layer. In one embodiment, the matrix includes a cavity for receiving the coolant fluid. In one embodiment, the device further comprises a pump for recirculating the coolant fluid, the pump being in fluid communication with the cavity, the pump being in electrical communication with the controller which activates and deactivates the pump. In one embodiment, the device further comprises a container including a coolant fluid, wherein the container is in contact with the matrix or the light-emitting layer. In one embodiment, the coolant fluid is selected from biocompatible fluids. In one embodiment, the coolant fluid is a dielectric fluid.

The matrix (e.g., bandage) can contain a flexible plastic catheter enabling suction or irrigation of fluid. The addition of squeeze bulbs similar to Jackson-Pratt drains can be used to either irrigate, infuse, or suck out wound substances to further enable healing and mitigate microbial infection. The catheter inserted into the matrix can also be connected to mechanical pumps for suction or infusion.

In another aspect, the disclosure provides a kit for improving healing behavior of a biological wound. The kit comprises: a matrix dimensioned to cover the wound; at least two light-emitting layers, each light-emitting layer comprising a plurality of light sources, at least one of the light-emitting layers being configured to irradiate the wound or a region adjacent the wound with light at a first range of wavelengths, and at least another of the light-emitting layers being configured to irradiate the wound or the region adjacent the wound with light at a second range of wavelengths, the first range of wavelengths being different than the second range of wavelengths; and a controller configured to be placed in electrical communication with the light sources of each light-emitting layer, the controller being configured to execute a program stored in the controller to activate the light sources of each light-emitting layer to irradiate the wound or the region adjacent the wound when the device is placed over the wound.

In one embodiment, the kit further comprises at least one additional matrix dimensioned to cover the wound. In one embodiment, the first range of wavelengths ranges from 700 nanometers to 2000 nanometers, and the second range of wavelengths ranges from 380 nanometers to 700 nanometers. In one embodiment, the first range of wavelengths ranges from 700 nanometers to 900 nanometers, and the second range of wavelengths ranges from 380 nanometers to 410 nanometers.

In one embodiment, the kit further comprises a translucent film dimensioned to be positioned between the matrix and each light-emitting layer for connecting the matrix and at least one of the light-emitting layers. In one embodiment, the kit further comprises an antimicrobial adjuvant for synergistically potentiating antimicrobial activity of the light with respect to microbes on or adjacent the wound. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix. The matrix can include an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof.

In one embodiment, the matrix includes an associated bioactive agent selected from the group consisting of tetracyclines. In one embodiment, the matrix includes an associated bioactive agent selected from the group consisting of glycopeptide antibiotics. In one embodiment, the matrix has a surface including a channel, the surface facing the wound when the device is placed over the wound. In one embodiment, the kit includes a source of irrigation fluid that is configured to be placed in fluid communication with an inlet of the channel.

The range of wavelengths transmitted by the LEDs can range from 400-470 nm; from 600-700 nm, from 700-800 nm, from 800-1300 nm, comprising blue light, green light, red light, and near IR. The controller can be programmed to enable duty cycles ranging from 0% to 99% “on”; the matrix (e.g., bandage) layer would be relatively transparent enabling light transmission and either capable or not of being complexed with adjuvant antimicrobial substances. Wavelengths with or without additional antimicrobial substances can be tailored to attack either polymicrobial infections, gram-positive bacterial infections, gram-negative bacterial infections, anaerobic or aerobic infections, mycobacterial and fungal or mold infections.

In another aspect, the disclosure provides a kit for improving healing behavior of a biological wound. The kit comprises: a plurality of matrix components, each matrix component having a first section and a second section, the first section of one matrix component being dimensioned to matingly engage the second section of an adjacent matrix component to form all or a part of a matrix dimensioned to cover the wound; a light-emitting layer comprising a plurality of light sources, the light-emitting layer being configured to irradiate the wound or a region adjacent the wound with light at a range of wavelengths; and a controller configured to be placed in electrical communication with the light sources of the light-emitting layer, the controller being configured to execute a program stored in the controller to activate the light sources of each light-emitting layer to irradiate the wound or the region adjacent the wound when the matrix is placed over the wound.

In one embodiment, the first section of the one matrix component comprises a tab extending outward from a side of the one matrix component, and the second section of the adjacent matrix comprises a recess dimensioned to matingly engage the tab of the one matrix component.

In one embodiment, the range of wavelengths ranges from 380 nanometers to 700 nanometers. In one embodiment, the range of wavelengths ranges 380 nanometers to 410 nanometers.

In one embodiment, the kit further comprises a translucent film dimensioned to be positioned between the matrix and the light-emitting layer for connecting the matrix and the light-emitting layer. In one embodiment, the kit further comprises an antimicrobial adjuvant for synergistically potentiating antimicrobial activity of the light with respect to microbes on or adjacent the wound. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix. In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics. In one embodiment, the matrix has a surface including a channel, the surface facing the wound when the device is placed over the wound. In one embodiment, the kit further comprises a source of irrigation fluid configured to be placed in fluid communication with an inlet of the channel.

A combination of wavelengths (e.g. antimicrobial blue light with a wavelength of 400 nm, additional LEDs in the near IR wavelengths of 800-880 nm, or even 1280 nm can provide a combination of activities. 400 nm blue light can provide antimicrobial activity; 800-880 nm can enhance the killing of microbes in wound biofilm, acting synergistically with 400 nm. The 800-880 nm would additionally stimulate the infected host's healing response, further providing enhanced healing. Finally, higher wavelength such as 1280 nm can enhance immune activity of the infected host, further enhancing wound healing.

In another aspect, the disclosure provides a kit for improving healing behavior of a biological wound. The kit comprises: a matrix dimensioned to cover the wound; a light-emitting layer comprising a plurality of light sources, the light-emitting layer being configured to irradiate the wound or a region adjacent the wound with light at a range of wavelengths including near IR to stimulate microbes in a biofilm, rendering them more sensitive to microbicidal wavelengths; a controller configured to be placed in electrical communication with the light sources of the light-emitting layer, the controller being configured to execute a program stored in the controller to activate the light sources of each light-emitting layer to irradiate the wound or the region adjacent the wound when the matrix is placed over the wound; and an antimicrobial adjuvant for synergistically potentiating antimicrobial activity of the light with respect to microbes on or adjacent the wound. The invention provides synergism of near IR stimulation of microbes to enhance the destruction by microbicidal wavelengths.

In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix. In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics.

In one embodiment, the matrix has a surface including a channel, the surface facing the wound when the device is placed over the wound. In one embodiment, the kit further comprises a source of irrigation fluid configured to be placed in fluid communication with an inlet of the channel.

In one embodiment, the range of wavelengths ranges from 380 nanometers to 700 nanometers. In one embodiment, the range of wavelengths ranges 380 nanometers to 410 nanometers.

In one embodiment, the kit further comprises a translucent film dimensioned to be positioned between the matrix and the light-emitting layer for connecting the matrix and the light-emitting layer.

Adjuvant antimicrobial substances can comprise vitamin K₃ or menadione; tetracycline antibiotics which are activated by light; naturally occurring essential oils such as carvacrol; photo sensitizers such as methylene blue, with or without potassium iodide, EDTA can be included as an adjuvant. Additional photosensitizers such as chlorin E6 and ALA can be used.

In another aspect, the disclosure provides a method for improving healing behavior of a biological wound. The method comprises: (a) placing an antimicrobial adjuvant on or adjacent the wound; (b) covering at least a portion of the wound with a matrix; (c) irradiating the wound or a region adjacent the wound with light for a period of time, wherein the antimicrobial adjuvant synergistically potentiates antimicrobial activity of the light with respect to microbes on or adjacent the wound.

In one embodiment, the light has a wavelength that ranges from 380 nanometers to 500 nanometers. In one embodiment, the light has a wavelength that ranges from 380 nanometers to 700 nanometers. In one embodiment, the light is provided at a radiant exposure of at least 50 J/cm² during the period of time.

In one embodiment, step (c) comprises: (i) irradiating the wound or the region adjacent the wound with light at a first range of wavelengths, and (ii) irradiating the wound or the region adjacent the wound with light at a second range of wavelengths, the first range of wavelengths being different than the second range of wavelengths.

In one embodiment, the first range of wavelengths ranges from 700 nanometers to 2000 nanometers, and the second range of wavelengths ranges from 380 nanometers to 700 nanometers. In one embodiment, irradiating the wound or the region adjacent the wound with light at the second range of wavelengths begins after irradiating the wound or the region adjacent the wound with light at the first range of wavelengths.

In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix. In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics.

In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

The 800-880 nm wavelengths can additionally stimulate the infected host's healing response, further providing enhanced healing. Finally, higher wavelengths such as 1280 nm can enhance immune activity of the infected host, further enhancing wound healing.

In another aspect, the disclosure provides a method for improving healing behavior of a biological wound. The method comprises: (a) covering at least a portion of the wound with a matrix having surface including a channel, the surface facing the wound when the matrix covers at least the portion of the wound; (b) irradiating the wound or a region adjacent the wound with light for a period of time; and (c) feeding an irrigation fluid to an inlet of the channel to irrigate the wound.

In one embodiment, the light has a wavelength that ranges from 380 nanometers to 500 nanometers. In one embodiment, the light has a wavelength that ranges from 380 nanometers to 700 nanometers. In one embodiment, the light is provided at a radiant exposure of at least 50 J/cm² during the period of time.

In one embodiment, step (b) comprises: (i) irradiating the wound or the region adjacent the wound with light at a first range of wavelengths, and (ii) irradiating the wound or the region adjacent the wound with light at a second range of wavelengths, the first range of wavelengths being different than the second range of wavelengths. In one embodiment, step (a) comprises placing an antimicrobial adjuvant on or adjacent the wound, wherein the antimicrobial adjuvant synergistically potentiates antimicrobial activity of the light with respect to microbes on or adjacent the wound.

In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, the antimicrobial adjuvant is associated with the matrix. In one embodiment, the matrix includes an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics.

In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

Adjuvant antimicrobial substances can comprise vitamin K³ or menadione; tetracycline antibiotics which are activated by light; naturally occurring essential oils such as carvacrol; photo sensitizers such as methylene blue, with or without potassium iodide, EDTA can be included as an adjuvant. Additional photosensitizers such as chlorin E6 and ALA can be used.

In another aspect, the disclosure provides a method for killing or inhibiting growth of microbes in a biofilm. The method comprises: (a) placing an antimicrobial adjuvant on or adjacent the biofilm; (b) irradiating the wound or a region adjacent the biofilm with light for a period of time, wherein the antimicrobial adjuvant synergistically potentiates antimicrobial activity of the light with respect to microbes on or adjacent the biofilm.

In one embodiment, the light has a wavelength that ranges from 380 nanometers to 700 nanometers. In one embodiment, step (b) comprises: (i) irradiating the biofilm or the region adjacent the biofilm with light at a first range of wavelengths, and (ii) irradiating the biofilm or the region adjacent the biofilm with light at a second range of wavelengths, the first range of wavelengths being different than the second range of wavelengths. In one embodiment, the first range of wavelengths ranges from 700 nanometers to 2000 nanometers, and the second range of wavelengths ranges from 380 nanometers to 700 nanometers. In one embodiment, irradiating the biofilm or the region adjacent the biofilm with light at the second range of wavelengths begins after irradiating the biofilm or the region adjacent the biofilm with light at the first range of wavelengths. In one embodiment, the light is provided at a radiant exposure of at least 50 J/cm² during the period of time.

In one embodiment, step (a) comprises placing an antimicrobial adjuvant on or adjacent the biofilm, wherein the antimicrobial adjuvant synergistically potentiates antimicrobial activity of the light with respect to microbes on or adjacent the biofilm. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, step (a) comprises covering at least a portion of the biofilm with a matrix. In one embodiment, the antimicrobial adjuvant is associated with the matrix. The matrix can include an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics.

In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

In one embodiment, the microbes are bacteria, fungus, or mold on or adjacent a biological wound. In one embodiment, the bacteria are methicillin-resistant Staphylococcus aureus (MRSA). In one embodiment, the bacteria are Escherichia coli. In one embodiment, the bacteria are Pseudomonas aeruginosa.

Adjuvant antimicrobial substances can comprise vitamin K³ or menadione; tetracycline antibiotics which are activated by light; naturally occurring essential oils such as carvacrol; photo sensitizers such as methylene blue, with or without potassium iodide, EDTA can be included as an adjuvant. Additional photosensitizers such as chlorin E6 and ALA can be used.

Additionally, the combination of near infrared (e.g., 800-890 nm) stimulates microbial metabolic activity in biofilms, potentially enhancing the activity of cidal antibiotics, further aiding in antimicrobial activity.

In another aspect, the disclosure provides a method for killing or inhibiting growth of microbes in a biofilm. The method comprises: (a) covering at least a portion of the biofilm with a matrix having surface including a channel, the surface facing the biofilm when the matrix covers at least the portion of the biofilm; (b) irradiating the biofilm or a region adjacent the biofilm with light for a period of time; and (c) feeding an irrigation fluid to an inlet of the channel to irrigate the biofilm.

In one embodiment, the light has a wavelength that ranges from 380 nanometers to 700 nanometers. In one embodiment, step (b) comprises: (i) irradiating the biofilm or the region adjacent the biofilm with light at a first range of wavelengths, and (ii) irradiating the biofilm or the region adjacent the biofilm with light at a second range of wavelengths, the first range of wavelengths being different than the second range of wavelengths. In one embodiment, the first range of wavelengths ranges from 700 nanometers to 2000 nanometers, and the second range of wavelengths ranges from 380 nanometers to 700 nanometers. In one embodiment, irradiating the biofilm or the region adjacent the biofilm with light at the second range of wavelengths begins after irradiating the biofilm or the region adjacent the biofilm with light at the first range of wavelengths. In one embodiment, the light is provided at a radiant exposure of at least 50 J/cm² during the period of time.

In one embodiment, step (a) comprises placing an antimicrobial adjuvant on or adjacent the biofilm, wherein the antimicrobial adjuvant synergistically potentiates antimicrobial activity of the light with respect to microbes on or adjacent the biofilm. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone. In one embodiment, step (a) comprises covering at least a portion of the biofilm with a matrix. In one embodiment, the antimicrobial adjuvant is associated with the matrix. The matrix can include an associated bioactive agent selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the matrix includes an associated bioactive agent selected from the group consisting of tetracyclines. In one embodiment, the matrix includes an associated bioactive agent selected from the group consisting of glycopeptide antibiotics.

In one embodiment, the irrigation fluid comprises an additive. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. In one embodiment, the irrigation fluid comprises oxygen.

In one embodiment, the microbes are bacteria, fungus, or mold on or adjacent a biological wound. In one embodiment, the bacteria are methicillin-resistant Staphylococcus aureus (MRSA). In one embodiment, the bacteria are Escherichia coli. In one embodiment, the bacteria are Pseudomonas aeruginosa.

Adjuvant antimicrobial substances can comprise vitamin K³ or menadione; tetracycline antibiotics which are activated by light; naturally occurring essential oils such as carvacrol; photo sensitizers such as methylene blue, with or without potassium iodide, EDTA can be included as an adjuvant. Additional photosensitizers such as chlorin E6 and ALA can be used.

Additionally, the combination of near infrared (e.g., 800-890 nm) stimulates microbial metabolic activity in biofilms, potentially enhancing the activity of cidal antibiotics, further aiding in antimicrobial activity.

It is an advantage of the invention to provide devices for improving healing behavior of a biological wound. The ability to use an LED layer with either 800-880 nm, or 1280 nm, or both would improve host wound healing of the wound. The lower wavelength enhances tissue regrowth and healing, while the higher wavelength stimulates immune cell function, both serving to improve wound healing.

It is another advantage of the invention to provide methods for improving healing behavior of a biological wound.

It is another advantage of the invention to provide kits for improving healing behavior of a biological wound. The ability to use an LED layer with either 800-880 nm, or 1280 nm, or both would improve host wound healing of the wound. The lower wavelength enhances tissue regrowth and healing, while the higher wavelength stimulates immune cell function, both serving to improve wound healing.

It is another advantage of the invention to provide methods for killing or inhibiting growth of microbes in a biofilm such as bacteria in a biofilm on or adjacent a biological wound.

It is another advantage of the invention to provide a device for combining both antimicrobial light therapy and light-enhanced wound and inflammation healing.

These and other features, aspects and advantages of various embodiments of the present invention will become better understood with regard to the following description, appended claims, and accompanying Figures.

### BRIEF DESCRIPTION OF THE DRAWINGS

FIG. 1 is an exploded perspective view of one embodiment of a device according to the invention for improving healing behavior of a biological wound.

FIG. 2 is a cross-sectional view of the device of FIG. 1 taken along line 2-2 of FIG. 1.

FIG. 3A is a side view of another embodiment of a device according to the invention for improving healing behavior of a biological wound.

FIG. 3B is a lateral view of the device of FIG. 3A on the forearm of a human subject.

FIG. 4 is a perspective view of another embodiment of a matrix suitable for use in a device according to the invention for improving healing behavior of a biological wound.

FIG. 5 is a top perspective view of a matrix component suitable for assembling a matrix suitable for use in a device according to the invention for improving healing behavior of a biological wound.

FIG. 6 is a bottom perspective view of the matrix component of FIG. 5.

FIG. 7 is a bottom view of the light emitting layer of the device of FIG. 3A.

FIG. 8 is a perspective view of a light emitting layer of the device of FIG. 10.

FIG. 9 is a perspective view of the light emitting layer of FIG. 8 and a matrix of the device of FIG. 10.

FIG. 10 is a perspective view of another embodiment of a device according to the invention for improving healing behavior of a biological wound.

FIG. 11 is a side view of another embodiment of a device according to the invention for improving healing behavior of a biological wound.

FIG. 12A shows a chemical structure of menadione (2-methyl-1,4-naphthoquinone).

FIG. 12B shows an absorption spectrum of menadione (400 M) in PBS containing either 1% DMSO or 1% DMSO/Tween, with maximum wavelength in 340 nm.

FIG. 13 shows a bar graph illustrating the log₁₀ colony forming unit (CFU/mL) reduction of 48-hour Staphylococcus aureus (MRSA) biofilm, following exposure to antimicrobial blue light (aBL) (250 J/cm²) in the presence of different concentrations of vancomycin (5, 15, 50, 500 μg/mL). Untreated control and colonies treated with aBL alone were also included. Error bars: standard error of the mean.

FIG. 14A shows inactivation kinetic curves illustrating the log₁₀ colony forming unit (CFU/mL) reduction of Planktonic cultures in the presence or absence of antimicrobial blue light (aBL) (50 mW/cm²-250 J/cm²) with the addition of different concentrations of menadione in bacterial strain Staphylococcus aureus (MRSA) USA 300 LUX.

FIG. 14B shows inactivation kinetic curves illustrating the log₁₀ colony forming unit (CFU/mL) reduction of Planktonic cultures in the presence or absence of antimicrobial blue light (aBL) (50 mW/cm²-250 J/cm²) with the addition of different concentrations of menadione in bacterial strain Pseudomonas aeruginosa 01.

FIG. 14C shows inactivation kinetic curves illustrating the log₁₀ colony forming unit (CFU/mL) reduction of Planktonic cultures in the presence or absence of antimicrobial blue light (aBL) (50 mW/cm²-250 J/cm²) with the addition of different concentrations of menadione in bacterial strain Escherichia coli ATCC2542.

FIG. 15A shows a bar graph illustrating the log₁₀ colony forming unit (CFU/mL) reduction of 48 hours biofilm cultures after treatment with the addition of different concentrations of menadione in MRSA in the absence of antimicrobial blue light (aBL).

FIG. 15B shows a bar graph illustrating the log₁₀ colony forming unit (CFU/mL) reduction of 48 hours biofilm cultures after treatment with the addition of different concentrations of menadione in MRSA in the presence of antimicrobial blue light (aBL) (50 mW/cm²-250 J/cm²).

FIG. 16A shows a bar graph illustrating the log₁₀ colony forming unit (CFU/mL) reduction of 48 hours biofilm cultures after treatment with the addition of different concentrations of menadione in Pseudomonas aeruginosa in the absence of antimicrobial blue light (aBL).

FIG. 16B shows a bar graph illustrating the log₁₀ colony forming unit (CFU/mL) reduction of 48 hours biofilm cultures after treatment with the addition of different concentrations of menadione in Pseudomonas aeruginosa in the presence of antimicrobial blue light (aBL) (50 mW/cm²-250 J/cm²).

FIG. 17A shows a bar graph illustrating the log₁₀ colony forming unit (CFU/mL) reduction of 48 hours biofilm cultures after treatment with the addition of different concentrations of menadione in Escherichia coli in the absence of antimicrobial blue light (aBL) (50 mW/cm²-250 J/cm²).

FIG. 17B shows a bar graph illustrating the log₁₀ colony forming unit (CFU/mL) reduction of 48 hours biofilm cultures after treatment with the addition of different concentrations of menadione in Escherichia coli in the presence of antimicrobial blue light (aBL) (50 mW/cm²-250 J/cm²).

FIG. 18A shows a bar graph illustrating the log₁₀ colony forming unit (CFU/mL) reduction of 48 hours of bacterial biofilms in Staphylococcus aureus (MRSA) in the wound beds in the ex-vivo porcine skin explants with different menadione concentrations in the absence of antimicrobial blue light (aBL).

FIG. 18B shows a bar graph illustrating the log₁₀ colony forming unit (CFU/mL) reduction of 48 hours of bacterial biofilms in Staphylococcus aureus (MRSA) in the wound beds in the ex-vivo porcine skin explants with different menadione concentrations in the presence of antibacterial blue light (aBL) (50 mW/cm²-250 J/cm²).

FIG. 19A shows a bar graph illustrating the log₁₀ colony forming unit (CFU/mL) reduction of 48 hours of bacterial biofilms in Pseudomonas aeruginosa in the wound beds in the ex-vivo porcine skin explants with different menadione concentrations in the absence of antimicrobial blue light (aBL).

FIG. 19B shows a bar graph illustrating the log₁₀ colony forming unit (CFU/mL) reduction of 48 hours of bacterial biofilms in Pseudomonas aeruginosa in the wound beds in the ex-vivo porcine skin explants with different menadione concentrations in the presence of antimicrobial blue light (aBL) (50 mW/cm²-250 J/cm²).

FIG. 20A shows a bar graph illustrating the log₁₀ colony forming unit (CFU/mL) reduction of 48 hours of bacterial biofilms in Escherichia coli in the wound beds in the ex-vivo porcine skin explants with different menadione concentrations in the absence of antimicrobial blue light (aBL).

FIG. 20B a bar graph illustrating the log₁₀ colony forming unit (CFU/mL) reduction of 48 hours of bacterial biofilms in Escherichia coli in the wound beds in the ex-vivo porcine skin explants with different menadione concentrations in the presence of antimicrobial blue light (aBL) (50 mW/cm²-250 J/cm²).

FIG. 21A shows changes in reactive oxygen species levels by the production of Reactive Oxygen Species (ROS) by menadione combined with blue light using a probe to specific ROS: DCF-DA measuring general ROS.

FIG. 21B shows changes in reactive oxygen species levels by the production of ROS by menadione combined with blue light using a probe to specific ROS: SOSG specific for singlet oxygen.

FIG. 21C shows changes in reactive oxygen species levels by the production of ROS by menadione combined with blue light using a probe to specific ROS: HPF specific for hydroxyl radical.

FIG. 22 shows a representation of a Jablonski diagram that illustrates the electronic states of energy and the transition between them. The diagram shows transitions between energy states that occur from the exposure of a molecule to a particular wavelength of light. The diagram illustrates that electron transfer or energy transfer can occur due to this exposure.

FIG. 23 shows the formation of a biofilm.

FIG. 24 shows a chemical structures of example tetracyclines (minocycline, doxycycline, and demeclocycline) useful in the present invention.

FIG. 25 shows bar graphs illustrating the log₁₀ colony forming unit (CFU/mL) reduction of 48 hours of bacterial biofilms in MRSA with different minocycline, doxycycline, and demeclocycline concentrations in the absence and the presence of antimicrobial blue light (aBL).

FIG. 26 shows bar graphs illustrating the log₁₀ colony forming unit (CFU/mL) reduction of 48 hours of bacterial biofilms in Escherichia coli with different minocycline, doxycycline, and demeclocycline concentrations in the absence and the presence of antimicrobial blue light (aBL).

FIG. 27 shows bar graphs illustrating the log₁₀ colony forming unit (CFU/mL) reduction of 48 hours of bacterial biofilms in Pseudomonas aeruginosa with different minocycline, doxycycline, and demeclocycline concentrations in the absence and the presence of antimicrobial blue light (aBL).

FIG. 28 shows changes in reactive oxygen species levels by the production of ROS by minocycline combined with blue light (left) and doxycycline combined with blue light (right) in MRSA using a DCF probe to specific ROS.

FIG. 29 shows changes in reactive oxygen species levels by the production of ROS by near infrared (NIR) radiation at 850 nm in a MRSA biofilm using a DCF probe to specific ROS (left) and using a SOSG probe to specific ROS.

FIG. 30 shows a bar graph illustrating the log₁₀ colony forming unit (CFU/mL) reduction of 48 hours of bacterial biofilms in MRSA in the absence and the presence of antibacterial blue light (aBL), and with different radiant exposure of near infrared (NIR) radiation at 850 nm combined with treatment with antimicrobial blue light.

Like reference numerals will be used to refer to like parts from Figure to Figure in the following detailed description. Any drawings herein are not shown to scale. Where dimensions are given in the text or figures, these dimensions are merely example values which could be used with one or more example implementations and do not limit the scope of the disclosed invention.

### DETAILED DESCRIPTION OF THE INVENTION

The present disclosure provides methods and devices for improving healing behavior of a biological wound, kits for improving healing behavior of a biological wound, and methods for killing or inhibiting growth of microbes in a biofilm such as bacteria in a biofilm on or adjacent a biological wound.

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Furthermore, relative terms, such as “lower” or “bottom,” “upper” or “top,” “left” or “right,” “above” or “below,” “front” or “rear,” may be used herein to describe one element's relationship to another element as illustrated in the Figures. It will be understood that relative terms are intended to encompass different orientations of the device in addition to the orientation depicted in the Figures.

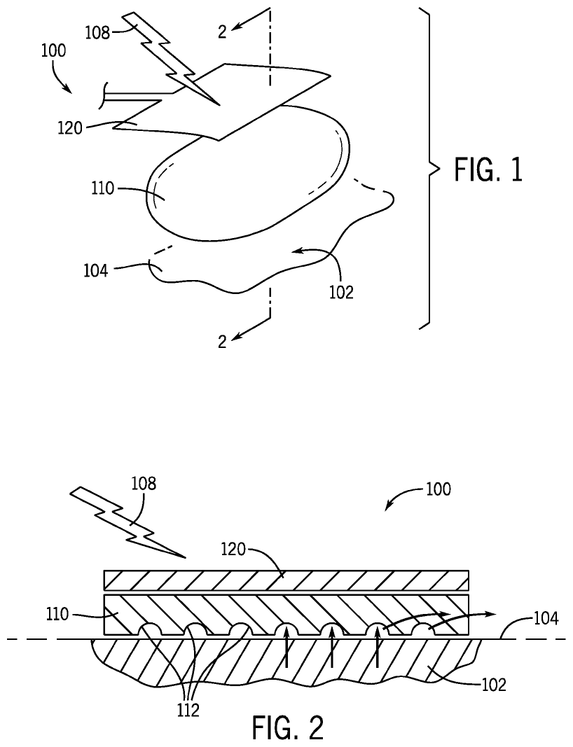

Referring now to FIGS. 1 and 2, there is shown one non-limiting example embodiment of a device 100 according to the invention for improving healing behavior of a biological wound 102 in a body part 104, such as a torso, head, neck, arm, or leg. The device 100 includes a matrix 110 dimensioned to cover the wound 102; a light-emitting layer 120 comprising a plurality of light sources; and a controller in electrical communication with the light sources. The controller is configured to execute a program stored in the controller to activate the light sources to irradiate the wound 102 or a region adjacent the wound 102 with light for a period of time when the device 100 is placed over the wound 102. In one embodiment of the device 100, the light 108 is light having a wavelength that ranges from 380 nanometers to 700 nanometers, or 380 nanometers to 420 nanometers, or 380 nanometers to 410 nanometers. The surface of the matrix 110 that faces the wound 102 when the device 100 is placed over the wound 102 includes channels 112 directed toward an interior of the matrix 110. The channels 112 can receive an irrigation fluid from a source of irrigation fluid, wherein the irrigation fluid enters an inlet of each channel, passes over the wound 102, and exits an outlet of each channel 112. The arrows in FIG. 2 shows a flow path of the irrigation fluid.

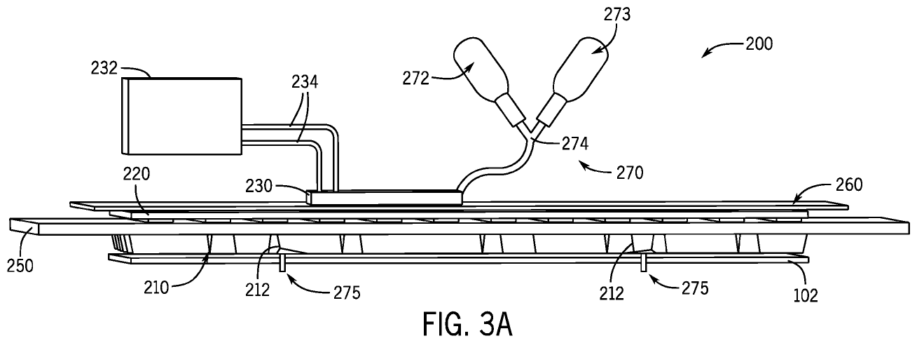

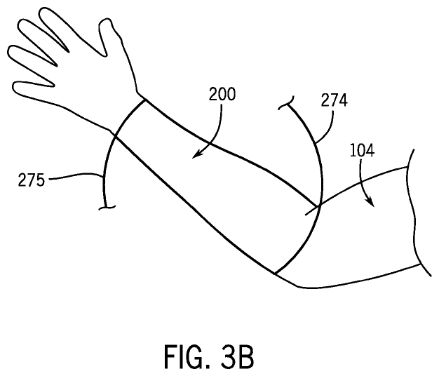

Turning now to FIGS. 3A and 3B, there is shown another non-limiting example embodiment of a device 200 according to the invention for improving healing behavior of a biological wound 102 in a body part 104, such the arm shown in FIG. 3B. The device 200 includes a matrix 210 dimensioned to cover the wound 102; a flexible light-emitting layer 220 comprising a plurality of light sources; and a controller 230 in electrical communication with the light sources. The controller 230 is powered by a battery 232 as a power source which is in electrical communication with the controller 230 via lines 234. The surface of the matrix 210 that faces the wound 102 when the device 200 is placed over the wound 102 includes channels 212 directed toward an interior of the matrix 210. The channels 212 can receive an irrigation fluid from sources 272, 273 of the irrigation fluid, wherein the irrigation fluid is supplied via conduit 274 to enters an inlet of each channel 212, passes over the wound 102, and exits an outlet of each channel 212 via conduit 275 that acts as a waste collector for the irrigation fluid. A translucent (preferably transparent) film 250 having double sided adhesive is positioned between the matrix 210 and the light-emitting layer 220 such that the film 250 connects the matrix 210 and the light-emitting layer 220. A cover sheet 260 is placed over the light-emitting layer 220.

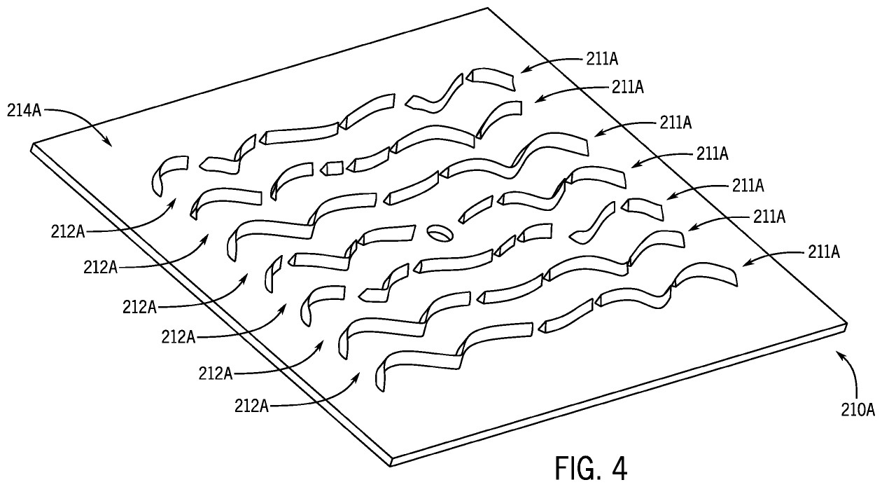

Looking at FIG. 4, there shown another embodiment of a matrix 210A suitable for use in the device 200. The matrix 210A includes walls 211A that extend away from a surface 214A of the matrix 210A that faces the wound 102 when the device 200 is placed over the wound 102. The walls 211A create channels 212A can receive an irrigation fluid from sources 272, 273 of the irrigation fluid, wherein the irrigation fluid is supplied via conduit 274 to enters an inlet (on the left side of FIG. 4) of each channel 212A, passes over the wound 102, and exits an outlet (on the right side of FIG. 4) of each channel 212A via conduit 275 that acts as a waste collector for the irrigation fluid.

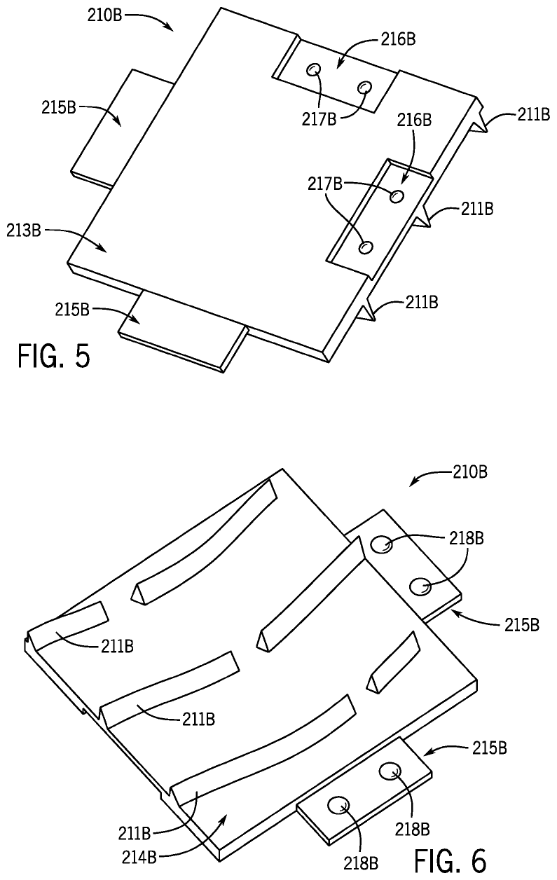

Referring now to FIGS. 5 and 6, there is shown a matrix component 210B suitable for assembling a matrix suitable for use in the device 200. The matrix component 210B includes a first surface 213B and a second surface 214B. The matrix component 210B includes walls 211B that extend away from the surface 214B of the matrix component 210B that faces the wound 102 when the device 200 is placed over the wound 102. The walls 211B create channels can receive an irrigation fluid from a source of the irrigation fluid as described above. The matrix component 210B has a first section 215B in the form of a tab and a second section 216B in the form of a recess. The first section 215B of one matrix component can be dimensioned to matingly engage the second section 216B of an adjacent matrix component to form all or a part of a matrix dimensioned to cover the wound. Depressions 217B in the second section 216B receive protrusions 218B from the first section 215B in an interference fit. In this manner, any number of matrix component 210B can be assembled together to form a larger matrix to cover larger wounds.

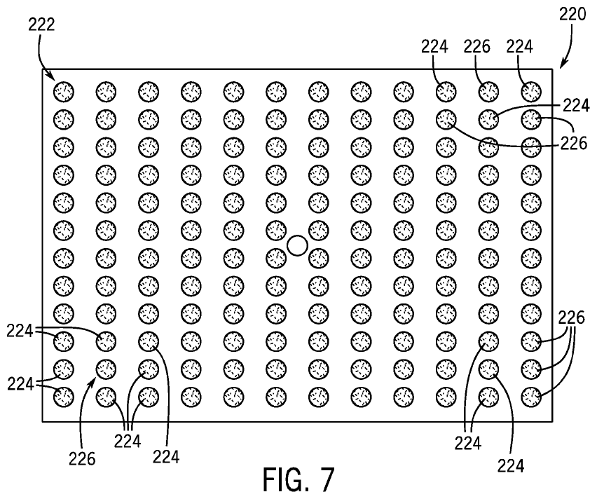

Looking now at FIG. 7, there is shown a non-limiting example embodiment of the light emitting layer 220 of the device 200 of FIG. 3A. The light emitting layer 220 has a surface 222 that faces the wound 102 when the device 200 is placed over the wound 102. An array of light-emitting diodes is arranged on the surface 222 of the light emitting layer 220.

Various configurations of the light-emitting diodes are possible. In the sub-array at the lower left corner of the light emitting layer 220, eight light-emitting diodes 224 surround a single light-emitting diode 226. The light-emitting diodes 224 emit light having a first range of wavelengths ranging from 700 nanometers to 2000 nanometers, or 700 nanometers to 1000 nanometers, or 700 nanometers to 900 nanometers. The light-emitting diode 226 emits light having a second range of wavelengths ranging from 380 nanometers to 700 nanometers, or 380 nanometers to 420 nanometers, or 380 nanometers to 410 nanometers. The sub-array pattern of eight light-emitting diodes 224 surrounding a single light-emitting diode 226 can be over the entire surface 222 of the light emitting layer 220.

In the sub-array at the lower right corner of the light emitting layer 220, alternating columns of light-emitting diodes 224 and light-emitting diodes 226 are used. The light-emitting diodes 224 emit light having a first range of wavelengths ranging from 700 nanometers to 2000 nanometers, or 700 nanometers to 1000 nanometers, or 700 nanometers to 900 nanometers. The light-emitting diodes 226 emit light having a second range of wavelengths ranging from 380 nanometers to 700 nanometers, or 380 nanometers to 420 nanometers, or 380 nanometers to 410 nanometers. The sub-array pattern of alternating columns of light-emitting diodes 224 and light-emitting diodes 226 can be over the entire surface 222 of the light emitting layer 220.

In the sub-array at the upper right corner of the light emitting layer 220, the light-emitting diodes 224 and light-emitting diodes 226 alternate in each row and column. The light-emitting diodes 224 emit light having a first range of wavelengths ranging from 700 nanometers to 2000 nanometers, or 700 nanometers to 1000 nanometers, or 700 nanometers to 900 nanometers. The light-emitting diodes 226 emit light having a second range of wavelengths ranging from 380 nanometers to 700 nanometers, or 380 nanometers to 420 nanometers, or 380 nanometers to 410 nanometers. The sub-array pattern of alternating in the rows and columns of light-emitting diodes 224 and light-emitting diodes 226 can be over the entire surface 222 of the light emitting layer 220.

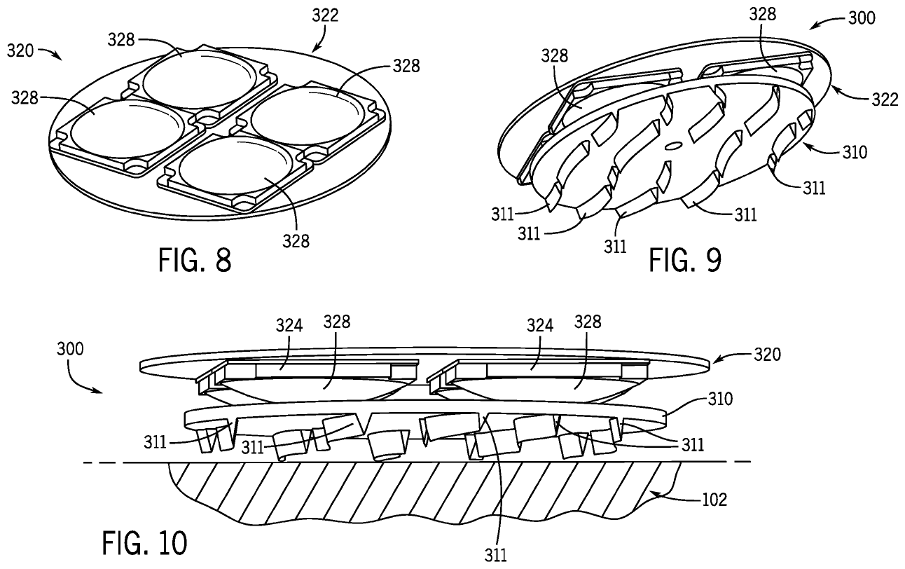

Turning now to FIGS. 8, 9 and 10, there is shown another non-limiting example embodiment of a device 300 according to the invention for improving healing behavior of a biological wound 102 in a body part. The device 300 includes a matrix 310 dimensioned to cover the wound 102; a flexible light-emitting layer 320 comprising a plurality of light sources; and a controller in electrical communication with the light sources. The light sources can comprise light-emitting diodes 324 and lenses 328 that focus or disperse the light emitted from the light-emitting diodes 324. The light-emitting diodes 324 can emit light having a first range of wavelengths ranging from 700 nanometers to 2000 nanometers, or 700 nanometers to 1000 nanometers, or 700 nanometers to 900 nanometers. Alternatively, the light-emitting diodes 326 can emit light having a second range of wavelengths ranging from 380 nanometers to 700 nanometers, or 380 nanometers to 420 nanometers, or 380 nanometers to 410 nanometers. The light-emitting diodes 326 provide sufficient light to the wound treatment site to improve healing of the wound, and may provide light flux between 10 mW/cm² and 60 mW/cm², preferably around 50 mW/cm². The controller can be configured to execute a program stored in the controller to activate the light-emitting diodes 326 to irradiate the wound 102 or a region adjacent the wound 102 with light for a period of time when the device 300 is placed over the wound 102 such that the light is provided at a radiant exposure of at least 50 J/cm², or at least 75 J/cm², or at least 100 J/cm², or at least 150 J/cm², or at least 200 J/cm², or at least 250 J/cm², during the period of time. The matrix 310 includes walls 311 that extend away from a surface of the matrix 310 that faces the wound 102 when the device 300 is placed over the wound 102. The walls 311 create channels can receive an irrigation fluid from source(s) of the irrigation fluid, wherein the irrigation fluid is supplied via a conduit to enter an inlet of each channel, passes over the wound 102, and exits an outlet of each channel via a conduit that acts as a waste collector for the irrigation fluid.

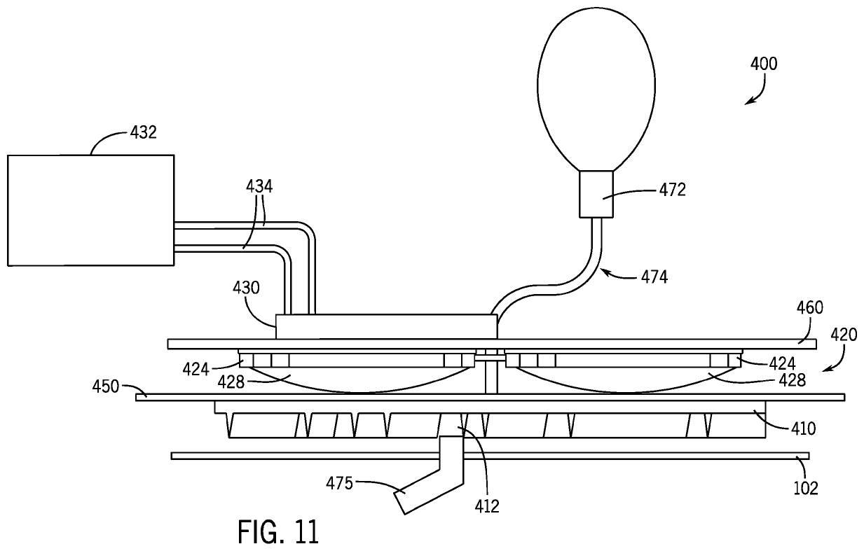

Referring now to FIG. 11, there is shown another non-limiting example embodiment of a device 400 according to the invention for improving healing behavior of a biological wound 102 in a body part 104. The device 400 includes a matrix 410 dimensioned to cover the wound 102; a flexible light-emitting layer 420 comprising a plurality of light sources; and a controller 430 in electrical communication with the light sources. The light sources can comprise light-emitting diodes 424 and lenses 428 that focus or disperse the light emitted from the light-emitting diodes 424. The controller 430 is powered by a battery 432 as a power source which is in electrical communication with the controller 430 via lines 434. The light-emitting diodes 424 can emit light having a first range of wavelengths ranging from 700 nanometers to 2000 nanometers, or 700 nanometers to 1000 nanometers, or 700 nanometers to 900 nanometers. Alternatively, the light-emitting diodes 426 can emit light having a second range of wavelengths ranging from 380 nanometers to 700 nanometers, or 380 nanometers to 420 nanometers, or 380 nanometers to 410 nanometers. The light-emitting diodes 426 provide sufficient light to the wound treatment site to improve healing of the wound, and may provide light flux between 10 mW/cm² and 60 mW/cm², preferably around 50 mW/cm². The controller can be configured to execute a program stored in the controller to activate the light-emitting diodes 426 to irradiate the wound 102 or a region adjacent the wound 102 with light for a period of time when the device 400 is placed over the wound 102 such that the light is provided at a radiant exposure of at least 50 J/cm², or at least 75 J/cm², or at least 100 J/cm², or at least 150 J/cm², or at least 200 J/cm², or at least 250 J/cm², during the period of time. The surface of the matrix 410 that faces the wound 102 when the device 400 is placed over the wound 102 includes a channel 412 directed toward an interior of the matrix 410. The channel 412 can receive an irrigation fluid from source 472 of the irrigation fluid, wherein the irrigation fluid is supplied via conduit 474 to enters an inlet of the channel 412, passes over the wound 102, and exits an outlet of the channel 412 via conduit 475 that acts as a waste collector for the irrigation fluid. A translucent (preferably transparent) film 450 having double sided adhesive is positioned between the matrix 410 and the light-emitting layer such that the film 450 connects the matrix 410 and the light-emitting layer. A cover sheet 460 is placed over the light-emitting layer.

Any of the matrices 110, 210, 210A, 310, and 410 or the matrix component 210B of the devices 110, 200, 300, and 400 may comprise a biocompatible material such as polyethylene, polypropylene, nylon, polyester, polyurethane, polyvinyl chloride, polyvinyl alcohol, polyvinylidene chloride, polyethylene/vinyl acetate copolymer, polyethylene/acrylic acid copolymer, polydimethylsiloxane (PDMS), and the like. The biocompatible material may be drug-eluting. Any of the matrices 110, 210, 210A, 310, and 410 of the devices 110, 200, 300, and 400 may have a thickness in a range of 0.5 millimeters to 20 millimeters, or 0.5 millimeters to 10 millimeters, or 2 millimeters to 8 millimeters.

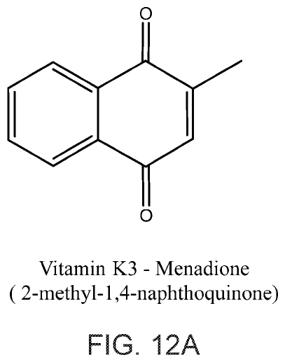

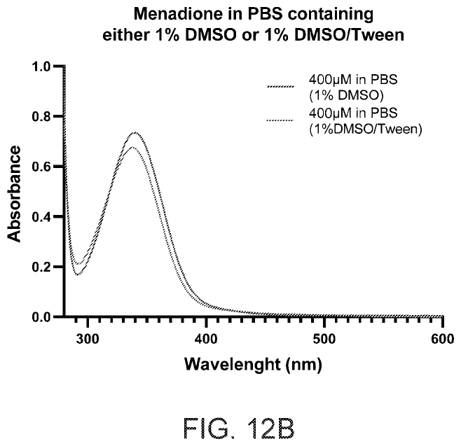

Any of the devices 100, 200, 300, or 400 may be used in one non-limiting example method according to the invention for improving healing behavior of a biological wound in a body part of a subject. The “subject” can be a mammal, preferably a human. In the method, an antimicrobial adjuvant is placed on or adjacent the wound 102, and at least a portion of the wound 102 is covered with a matrix (e.g., any of the matrices 110, 210, 210A, 310, and 410). Various methods can be used to place the antimicrobial adjuvant on or adjacent the wound 102. For example, the antimicrobial adjuvant may be directly applied on or adjacent the wound 102, and then at least a portion of the wound 102 is covered with a matrix of any of the devices 100, 200, 300, or 400. Alternatively, an antimicrobial adjuvant is “associated” with the matrix by being directly or indirectly, physically or chemically bound to the matrix. Non-limiting examples of chemical bonds include covalent bonds, ionic bonds, coordinate bonds, and hydrogen bonds. Indirect bonding can include the use of a group of atoms (i.e., a linker) that chemically links the bioactive agent and the matrix. Non-limiting examples of physical bonding include physical adsorption, absorption, and entrapment. The antimicrobial adjuvant can elute from an interior of the matrix to a location on or adjacent the wound, or from a surface of the matrix that faces the wound when the matrix covers the wound. The wound or a region adjacent the wound is irradiated with light from the light-emitting layer (e.g., any of the light-emitting layers 120, 220, 320, and 420 for a period of time, preferably for a period of time such that the light is provided at a radiant exposure of at least 50 J/cm² during the period of time. The antimicrobial adjuvant synergistically potentiates antimicrobial activity of the light with respect to microbes on or adjacent the wound. In one embodiment, the method does not require additional antimicrobial agents. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone (see FIG. 12A).

Any of the devices 100, 200, 300, or 400 may be used in another non-limiting example method according to the invention for improving healing behavior of a biological wound in a body part of a subject. In the method, the plurality of light sources comprises an array of light emitting diodes in electrical communication with the controller, and the controller is configured to execute a program stored in the controller to: (i) activate a first group of the light emitting diodes to irradiate the wound or the region adjacent the wound with light at a first range of wavelengths, and (ii) thereafter activate a second group of the light emitting diodes to irradiate the wound or the region adjacent the wound with light at a second range of wavelengths. The first range of wavelengths (e.g., 700-900 nanometers) is different than the second range of wavelengths (e.g., 380-410 nanometers), and the light at the first range of wavelengths functions as an antimicrobial adjuvant for the light at a second range of wavelengths.

Any of the devices 100, 200, 300, or 400 may be used in another non-limiting example method according to the invention for improving healing behavior of a biological wound in a body part of a subject. In the method, at least a portion of the wound 102 is covered with a matrix (e.g., any of the matrices 110, 210, 210A, 310, and 410), and the wound or a region adjacent the wound is irradiated with light from the light-emitting layer (e.g., any of the light-emitting layers 120, 220, 320, and 420 for a period of time, preferably for a period of time such that the light is provided at a radiant exposure of at least 50 J/cm² during the period of time. An irrigation fluid is fed to an inlet of one or more channels (e.g., any of channels 112, 212, 212A, or 412) of the matrix to irrigate the wound. An irrigation fluid is supplied via a conduit (e.g., 474 of device 400) and enters an inlet of a channel (e.g., 412 of device 400), and passes over the wound 102, and exits an outlet of the channel via a conduit (e.g., 475 of device 400) that acts as a waste collector for the used irrigation fluid. The irrigation fluid may comprise an additive in a carrier such as water. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. Alternatively, the irrigation fluid can comprise oxygen. Also, an issue using phototherapy is potential overheating on the skin caused by long periods of light exposure and power consumption for a portable device. In order to solve this problem, the irrigation fluid may be provided at a suitable temperature (e.g., 20-25° C.) to act as a coolant for the wound and adjacent skin.

Any of the devices 100, 200, 300, or 400 may be used in another non-limiting example method according to the invention for improving healing behavior of a biological wound in a body part of a subject in which an antimicrobial adjuvant, light irradiation, and a bioactive agent are used. Any of the matrices 110, 210, 210A, 310, and 410 or the matrix component 210B of the devices 110, 200, 300, and 400 may be associated with a bioactive agent. A bioactive agent is “associated” with the matrix if the bioactive agent is directly or indirectly, physically or chemically bound to the matrix. Non-limiting examples of chemical bonds include covalent bonds, ionic bonds, coordinate bonds, and hydrogen bonds. Indirect bonding can include the use of a group of atoms (i.e., a linker) that chemically links the bioactive agent and the matrix. Non-limiting examples of physical bonding include physical adsorption, absorption, and entrapment. The bioactive agent can elute from an interior of the matrix to a location on or adjacent the wound, or from a surface of the matrix that faces the wound when the matrix covers the wound.

A “bioactive agent” as used herein includes, without limitation, physiologically or pharmacologically active substances that act locally or systemically in the body. A bioactive agent is a substance used for the treatment, prevention, diagnosis, cure or mitigation of disease or illness, or a substance which affects the structure or function of the body or which becomes biologically active or more active after it has been placed in a predetermined physiological environment. Bioactive agents include, without limitation, cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, and therapeutics.

In one example embodiment, the matrix includes an associated bioactive agent selected from the group consisting of tetracyclines. In one example embodiment, the matrix includes an associated bioactive agent selected from minocycline, doxycycline, demeclocycline, and mixtures thereof. In one example embodiment, the matrix includes an associated bioactive agent selected from the group consisting of glycopeptide antibiotics, such as vancomycin.

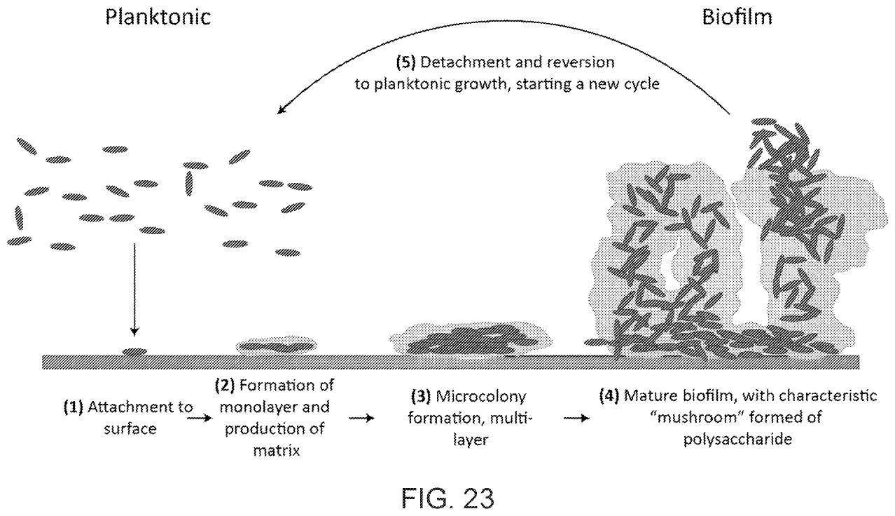

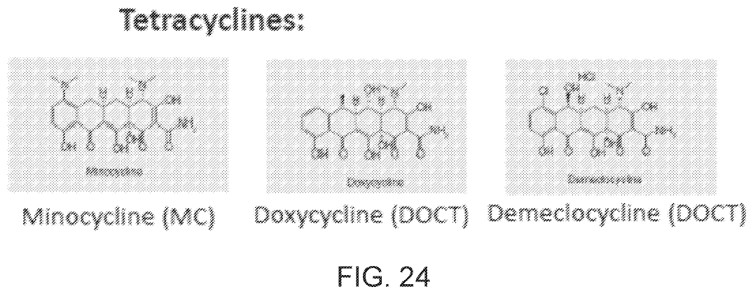

Any of the devices 100, 200, 300, or 400 may be used in one non-limiting example method according to the invention for killing or inhibiting growth of microbes in a biofilm. Looking at FIG. 23, a biofilm is an assemblage of surface-associated microbial cells that become enclosed in an extracellular polymeric substance matrix. The formation begins with a reversible attachment of the planktonic cells (brown ovals) followed by the adhesion to the surface (grey) (1). The bacteria then form a monolayer and irreversibly attach by producing an extracellular matrix (2). Next, a microcolony is formed where multilayers appear (3). During later stages, the biofilm is mature, forming characteristic “mushroom” structures due the polysaccharides (4). Finally, some cells start to detach and the biofilm (shown in yellow) will disperse (5). In the method, an antimicrobial adjuvant is placed on or adjacent the biofilm, and at least a portion of the biofilm is covered with a matrix (e.g., any of the matrices 110, 210, 210A, 310, and 410). Various methods can be used to place the antimicrobial adjuvant on or adjacent the biofilm. For example, the antimicrobial adjuvant may be directly applied on or adjacent the biofilm, and then at least a portion of the biofilm is covered with a matrix of any of the devices 100, 200, 300, or 400. Alternatively, an antimicrobial adjuvant is “associated” with the matrix by being directly or indirectly, physically or chemically bound to the matrix. The antimicrobial adjuvant can elute from an interior of the matrix to a location on or adjacent the biofilm, or from a surface of the matrix that faces the biofilm when the matrix covers the biofilm. The biofilm or a region adjacent the biofilm is irradiated with light from the light-emitting layer (e.g., any of the light-emitting layers 120, 220, 320, and 420 for a period of time, preferably for a period of time such that the light is provided at a radiant exposure of at least 50 J/cm² during the period of time. The antimicrobial adjuvant synergistically potentiates antimicrobial activity of the light with respect to microbes on or adjacent the biofilm. The microbes can be bacteria on or adjacent a biological wound of a subject. The bacteria can be methicillin-resistant Staphylococcus aureus (MRSA), or Escherichia coli, or Pseudomonas aeruginosa. In one embodiment, the antimicrobial adjuvant comprises a substituted naphthalene. In one embodiment, the antimicrobial adjuvant comprises an oxygen substituted naphthalene. In one embodiment, the antimicrobial adjuvant comprises 2-methyl-1,4-naphthoquinone (see FIG. 12A).

Any of the devices 100, 200, 300, or 400 may be used in another non-limiting example method according to the invention for killing or inhibiting growth of microbes in a biofilm. In the method, the plurality of light sources comprises an array of light emitting diodes in electrical communication with the controller, and the controller is configured to execute a program stored in the controller to: (i) activate a first group of the light emitting diodes to irradiate the biofilm or the region adjacent the biofilm with light at a first range of wavelengths, and (ii) thereafter activate a second group of the light emitting diodes to irradiate the biofilm or the region adjacent the biofilm with light at a second range of wavelengths. The first range of wavelengths (e.g., 700-900 nanometers) is different than the second range of wavelengths (e.g., 380-410 nanometers), and the light at the first range of wavelengths functions as an antimicrobial adjuvant for the light at a second range of wavelengths.

Any of the devices 100, 200, 300, or 400 may be used in another non-limiting example method according to the invention for killing or inhibiting growth of microbes in a biofilm. In the method, at least a portion of the biofilm 102 is covered with a matrix (e.g., any of the matrices 110, 210, 210A, 310, and 410), and the biofilm or a region adjacent the biofilm is irradiated with light from the light-emitting layer (e.g., any of the light-emitting layers 120, 220, 320, and 420 for a period of time, preferably for a period of time such that the light is provided at a radiant exposure of at least 50 J/cm² during the period of time. An irrigation fluid is fed to an inlet of one or more channels (e.g., any of channels 112, 212, 212A, or 412) of the matrix to irrigate the biofilm. An irrigation fluid is supplied via a conduit (e.g., 474 of device 400) and enters an inlet of a channel (e.g., 412 of device 400), and passes over the biofilm, and exits an outlet of the channel via a conduit (e.g., 475 of device 400) that acts as a waste collector for the used irrigation fluid. The irrigation fluid may comprise an additive in a carrier such as water. The additive can be selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof. Alternatively, the irrigation fluid can comprise oxygen.

Embodiments of the invention, such as the non-limiting example embodiments of the devices 100, 200, 300, or 400, may use a flexible optical fiber dressing system with a blue light source, and may keep colony counts below 105/gram of tissue, thereby reducing bacterial bioburden. Beneficial features of some embodiments of the invention may include: Optional features for particular embodiments may include: not harmful to host tissue (able to “breath”); able to handle exudate without “fouling”; broad antimicrobial effect (gram positive and gram negative pathogens); highly transparent; flexible and capable of being placed into irregular spaces; inexpensive components, simple manufacture; FDA approved materials; light weight; and battery-operated. Device considerations include: power density and heat dissipation. For the biology of infection, the device is intended to prevent and treat wound contamination and infection, and reduce bacterial bioburden and wound infections rapidly becoming biofilm.

### EXAMPLES

The following Examples are provided to demonstrate and further illustrate certain embodiments and aspects of the present invention and are not to be construed as limiting the scope of the invention.

### Example 1

In this Example 1, we show that Vitamin K₃ (menadione) acts as a photosensitizer to synergize with blue light to kill drug-resistant bacteria in biofilms.

### 1.1 Introduction

Antimicrobial resistance (AMR) is a threat to global human health of paramount significance, threatening to once again return common bacterial infections to major causes of death [Ref. 2-4]. It is universally acknowledged that the prolonged use, and overuse, of antibiotics in many settings is a major contributor [Ref. 2-4].

In a study of thousands of wound infections, in which antibiotics were antibiotic-susceptibility matched, treatment-induced resistance often resulted from rapid reinfection with a different strain, resistant to that antibiotic. The more prolonged and chronic the treatment, the more likely the development of that resistance. Complicating wound treatment is the fact that wound infections rapidly develop into biofilm infections, in which bacteria are encased in a matrix or glycocalyx of bacterial carbohydrates, bacterial and host DNA, bacterial and host proteins, all further rendering biofilm-resident bacteria dramatically less sensitive to antibiotics [Ref. 26] In fact, bacteria in biofilms are 100-1000 times less sensitive to antibiotics [Ref. 27]. Thus, alternatives to antibiotics, or ways of augmenting their activity, are becoming a necessity. Unfortunately, phototherapy is often conspicuously omitted as a technology to reduce bacterial infection while avoiding the development of antibiotic resistance [Ref. 4].

Many wound infections, especially cutaneous infections, are accessible to light. Light as a therapy against microbial infection was discovered over 100 years ago. More recently, blue light, in wavelengths from 400-470 nm, have been found to be effective in experimental biofilm infections both in vitro and in vivo [Ref. 6, 27]. It is now well-established that blue light, particularly at a wavelength of 405 nm, has potent in vitro and in vivo antimicrobial activities. These have been extensively reviewed [Ref. 6, 14]. At least one mechanism by which antimicrobial blue light (aBL) functions is through its ability to interact with endogenous chromophores within bacterial cells, especially intracellular porphyrins and flavins in the microbial cells. Though not fully understood, evidence supports the hypothesis that the absorption of photon energy of blue light by the bacterial chromophore results in the generation of reactive oxygen species, most notably singlet oxygen, superoxide, and hydrogen peroxide intracellularly in the bacteria, which do not have the same efficient antioxidant capabilities of mammalian cells. Depending on the bacterial species, microbial culture conditions, and the many variables of light administration (light/power density, total dose, environmental temperature, etc.), bactericidal activity in the realm of two-seven logs of killing may be achievable in vitro in planktonic cultures [Ref. 27]. Similar activities have been described in small animal models of infection, but no systematic studies have been carried out using antimicrobial blue light in large animals [Ref. 6].

A recent paper [Ref. 28] examining the effectiveness of antimicrobial therapy for diabetic foot infections found that 95% of the predominant pathogens were gram-positive cocci, the most virulent and resistant of which is methicillin-resistant Staphylococcus aureus, or MRSA. Cutaneous wound infections, including diabetic foot ulcers, are exceedingly common, often chronic, and commonly associated with biofilms that both protect the bacteria and further slow wound healing [Ref. 6]. Due to the very nature of these wounds, the combination of bacterial contamination and chronicity of infection makes prolonged antibiotic therapy currently inevitable. A landmark 2014 study in the U.S. [Ref. 3] involving over 32,000 matched pairs of diabetic patients, either with or without diabetic foot ulcers (DFU), found that the cost of DFU's alone contributed $9-$13 billion annually to the cost of diabetic care, and resulted in 4-5% of patients requiring amputation.

In this Example 1, we studied the effectiveness of aBL against MRSA, as well as other pathogens, in vitro and in an ex vivo porcine skin wound model (the closest animal simulant to human skin). The latter was chosen for preclinical screening [Ref. 29,30] before beginning large animal testing of blue light treatments for cutaneous wounds in vivo in swine, a necessary regulatory prelude to clinical trials. In the course of developing our ex vivo porcine skin model [Ref. 30] for evaluating phototherapy, we noted a substantial decrease in the bactericidal activity of aBL when tested in vitro and ex vivo in biofilms as compared with planktonic cultures.

Brynildsen et al. [Ref. 31] had previously described the use of menadione, or vitamin K₃, to augment bacterial intracellular ROS production. Menadione was reported to have multiple antimicrobial activities and was suggested as a potential topical agent [Ref. 9]. Menadione has been FDA approved for oral administration and is still widely included in animal feed as a dietary supplement. Menadione is a quinone, an analog of 1,4-naphthoquinone (see FIG. 12A) [Ref. 1]. It is also known as vitamin K₃, a fat-soluble pro-drug of vitamin K, which is converted to vitamin K₂ (menaquinone) in the liver. The essential role of vitamin K has been well known in blood coagulation and has been thought to be a regulatory nutrient in tissue calcification [Ref. 10]. However, at the high doses initially used orally (5 mg., 10 mg.) and intravenously (2 mg.), hepatitis was described in neonates, and these forms of the drug were withdrawn from the market. However, to us cutaneous application seemed a safe way to augment aBL-mediated ROS generation within bacteria, and thus to increase aBL-induced antimicrobial effects for use in cutaneous wounds, thus sparing the use of antibiotics.

### 1.2 Material and Methods

### 1.2.1 Absorption UV-Vis Spectroscopy

Stock solutions of menadione (10 mg/mL) were prepared in DMSO only and in DMSO/Tween (1:1) to improve the solubility. Different concentrations of menadione were prepared to make a calibration curve by diluting the stock solution in PBS so that the final solution was either 1% DMSO or 1% DMSO/Tween. The absorbance spectra were measured using an Evolution™ 300 UV-Vis Spectrophotometer (Thermo Scientific, Waltham, Massachusetts, USA). The calibration curve was measured, and molar absorption coefficient calculated.

### 1.2.2 Chemicals

Menadione (Mn), Dimethyl sulfoxide (DMSO), and Tween 80 were purchased from Sigma-Aldrich (St. Louis, Missouri, USA). Phosphate-buffered saline (PBS) for microbial cell suspension and serial dilution and Brain-heart infusion broth (BHI) were purchased from Fisher Scientific (Waltham, Massachusetts, USA). Menadione stock solution of 100 mg/mL was prepared by dissolving it in a mixture of DMSO and Tween 80 in the ratio of 1:1 and was diluted to the final concentration in PBS before use.

### 1.2.3 Blue Light Source and Light Measurements

As a light source for aBL irradiation, a light-emitting diode (LED; M405L4; Thorlabs, USA) with a peak emission of 405 nm and a full width at half maximum of 25 nm was used. The power density/irradiance (mW/cm²) of transmitted light energy was established by measuring the surface of the target with the use of a PM100D power meter (Thorlabs, USA). We used different power densities: 50, 30, 15 and 10 mW/cm². The radiant exposure (also referred to as fluence or dose) was calculated with the following equation (Radiant exposure (J/cm²)=Irradiance (W/cm²)×Exposure time(s)).

### 1.2.4 Bacterial Strains and Growth Conditions

The strains used in this study were methicillin-resistant Staphylococcus aureus (USA300 Lux, AF0003, IQ0064), Pseudomonas aeruginosa PA01 (P. aeruginosa) and Escherichia coli ATCC 25922 (E. coli). These strains were routinely cultured on BHI agar plates at 37° C. in 5% CO2.

### 1.2.5 Minimum Inhibitory Concentrations (MICs) for Menadione

The MICs of menadione in different bacterial strains were determined by a standard broth microdilution assay according to the Clinical and Laboratory Standards Institute. Briefly, menadione was prepared at 100 mg/mL in Tween 80/DMSO (1:1) as a stock solution. The stock solution was diluted to 2048 μg/mL in BHI and then serial dilutions were performed to 1024-0.015 μg/mL in a 96-well plate. Then, a 10 μL stationary growth-phase culture of 10⁸ CFU/mL bacteria in BHI broth was added to the 96-well plate containing the serial dilutions of menadione. The medium containing a similar amount of Tween 80/DMSO (1:1) served as a control. The microplates were incubated at 37° C. for 24 hours and the lowest concentration of the compound capable of completely inhibiting bacterial growth was referred to as the MIC.

### 1.2.6 Photoinactivation of Bacteria in Planktonic Cultures In Vitro by Menadione+Antimicrobial Blue Light

The bacterial cells were grown in BHI broth medium shaken overnight. Cells were collected by centrifugation at 4,000 rpm for 5 minutes and suspended in PBS at a density of 10⁸ colony-forming units (CFU)/mL (estimated by measuring the optical density at 600 nm; O.D 0.1=10⁸ cells/mL). The microbial suspension in PBS was transferred to a 35×12-mm dish and mixed with different concentrations of menadione: 0, 0.5, 1 and 2 μg/mL for MRSA USA300, AF003 and IQ0064; 0, 1, 10 and 30 μg/mL for P. aeruginosa PA01; and 0, 16, 32, 64 and 128 μg/mL for E. coli ATCC 25922. After incubation in the dark at 25° C. for 1 hour, the planktonic suspension of cells at room temperature was irradiated using aBL (405 nm) at 50 mW/cm² with different radiant exposures (0-250 J/cm²). During the aBL irradiation, 40 μL of samples were withdrawn every 15 minutes, and the CFUs were determined by 10-fold serial dilutions in PBS (10-1 to 10-7 dilution factors), plated on BHI agar plates as described previously [Ref. 17]. CFUs were counted after overnight incubation at 37° C. Experiments were performed in triplicate.

### 1.2.7 Photoinactivation of Bacteria in In Vitro Biofilms by Menadione+Antimicrobial Blue Light

The bacterial suspension was grown in a shaking incubator overnight in BHI broth. Cells were collected by centrifugation at 4,000 rpm for 5 minutes and suspended in PBS at a density of 106 CFU/mL. Then, the biofilms were grown for 48 hours (unless otherwise specified) in 96-well microtiter plates as described previously [Ref. 27], with a renewal of the media every 24 hours. Following the 48-hour biofilm growth, fresh 200 μL of PBS with or without menadione in different concentrations were added to the wells and the biofilms were incubated in the dark at 25° C. for 1 hour: 0, 0.5, 1, and 2 μg/mL for MRSA USA300, AF0001, IQ0064; 0, 1, 10, 30 and 50 μg/mL for P. aeruginosa PA01; and 0, 100, and 150 μg/mL for E. coli ATCC 25922. After that, the control and the menadione-treated biofilms were maintained without irradiation (“menadione/dark”). Simultaneously, the aBL was delivered to the other biofilm groups testing different power densities (50, 30, 15, and 10 mW/cm²) while keeping the same radiant exposure of 250 J/cm². After the light exposure, the biofilms were carefully washed two times with PBS and 200 μL of PBS was added to each well. Then, the bacterial biofilms in each well were harvested by scratching with a sterile pipette tip and three wells of each group were pooled together in a 1.5 mL microcentrifuge tube. The total volume obtained, 600 μL (3 wells), was sonicated using a Branson 2510 Water Bath Sonicator (Marshall Scientific, LLC) for 5 minutes and the CFU was determined by 10-fold serial dilutions in PBS on BHI agar plates. CFU was counted after overnight incubation at 37° C. Experiments were performed in triplicate.

### 1.2.8 Photoinactivation of Bacterial Biofilms in Ex Vivo Porcine Partial Thickness Wounds by Menadione+Antimicrobial Blue Light

Skin was handled and sanitized with 70% isopropanol solution after hair shaving. The explants and inner wounds were created using a 12 mm and 4 mm biopsy punches respectively. The explants were placed in 12 wells plates using a (8 μm pore) inserts (Corning™ Falcon™). 1 mL of DMEM media was added to each well in the plate before the insert. After 24 hours, the inner wounds in the explants were inoculated with 20 μL of 10⁸ CFU/mL of bacterial solution. After that, the explants were incubated at 37° C. and 5% CO2 for 48 hours to ensure the growth of a mature bacterial biofilm. The inner wounds of the explants were replenished daily with fresh BHI media to ensure that the wounds are hydrated.

### 1.2.9 Measurement of Reactive Oxygen Species (ROS)

1.2.9.1 General ROS measured by 2′,7′-dichlorofluorescein (DCFH-DA) assay: DCF-DA was purchased from Thermofisher. This probe is chemically reduced and acetylated in nonfluorescent form until the acetate groups are removed by intracellular esterase and oxidation [Ref. 22]. The production of ROS by menadione when combined with blue light was measured in the presence and absence of bacteria. The bacteria (MRSA) was grown in BHI broth medium with shaking overnight. Cells were collected by centrifugation at 4,000 rpm for 5 minutes and resuspended in PBS in the stationary growth phase culture of 1×10⁸ CFU/mL. The microbial suspension was transferred to a 35×12-mm dish, mixed with 10 μg/mL of menadione, and stained with 10 μM DCFH-DA solution per the manufacturer's instruction. The solutions (with and without bacteria) were irradiated with the blue light at 50 mW/cm² and the samples were collected every 15 minutes to be analyzed by Microplate Spectrofluorometer (SPECTRAmax®, Molecular Devices, USA) at λₑₓ=495 nm and λₑₘ=525 nm. The control and the menadione/dark groups were generated under the same conditions without irradiation.

1.2.9.2 Singlet oxygen ¹O₂ assay: The singlet oxygen was measured by Singlet Oxygen Sensor Green Reagent (SOSG) (Thermofisher). In the presence of singlet oxygen, SOSG emits a green fluorescence similar to that of fluorescein (excitation/emission maxima ˜504/525 nm). The MRSA were grown in BHI broth medium with shaking overnight. Cells were collected by centrifugation at 4,000 rpm for 5 minutes and resuspended in PBS in the stationary growth phase culture of 1×10⁸ CFU/mL. The microbial suspension was transferred to a 35×12-mm dish, mixed with 10 μg/mL of menadione, and stained with 10 μM SOSG solution per the manufacturer's instruction. The bacteria were irradiated with the blue light at 50 mW/cm² and the samples were collected each 15 minutes to be analyzed by Microplate Spectrofluorometer (SPECTRAmax®, Molecular Devices, USA) at λₑₓ=504 nm and λₑₘ=525 nm. The control and the menadione/dark groups were generated under the same conditions without irradiation.

1.2.9.3 Hydroxyl radical HO′ assay: The hydroxyl radical was measured by 3′-(p-hydroxyphenyl) fluorescein (HPF) (Thermofisher). HPF is a new fluorescein derivative, and it is nonfluorescent until reacted with the hydroxyl radical or peroxynitrite anion. The MRSA were grown in BHI broth medium with shaking overnight. Cells were collected by centrifugation at 4,000 rpm for 5 minutes and resuspended in PBS in the stationary growth phase culture of 1×10⁸ CFU/mL. The microbial suspension was transferred to a 35×12-mm dish, mixed with 10 μg/mL of menadione, and stained with 10 μM HPF solution per the manufacturer's instruction. The bacteria were irradiated with the blue light at 50 mW/cm² and the samples were collected each 15 minutes to be analyzed by Microplate Spectrofluorometer (SPECTRAmax®, Molecular Devices, USA) at λₑₓ=490 nm and λₑₘ=515 nm. The control and the menadione/dark groups were generated under the same conditions without irradiation.

### 1.3 Results

### 1.3.1 Spectroscopy

The chemical structure of the menadione is shown in FIG. 12A. The chemical structure of menadione is lipophilic and not soluble in water. Thus, we dissolved the menadione in two conditions: in PBS containing either 1% DMSO or 1% DMSO/Tween80 at 400 μM. Tween 80 is a surfactant and the idea is to mix with the DMSO before to add to PBS is due to necessity to improve the solubility of menadione in aqueous solution. We measured the absorption spectrum in both conditions to analyze if there is any change in optical properties. The range of wavelength absorption of menadione was measured between 200-800 nm (see FIG. 12B) to choose the optimum wavelength of the light absorption spectra. It was observed in PBS with 1% DMSO/Tween80, menadione presented the best solubility. The maximum wavelength of absorption found was 340 nm for both conditions. Despite of the Amax of menadione is in UVA light, the spectra showed an absorption in blue light. The molar absorptivity coefficient was compared in different wavelength according with the Table 1.

TABLE 1
Menadione UV-V absorption parameters at different wavelengths

| | ε (L · moL⁻¹ · cm⁻¹) | | |
|---|---|---|---|
| | λ = 340 nm | λ = 380 nm | λ = 405 nm |
| 1% DMSO in PBS | 2458 | 572.1 | 128.82 |
| 1% DMSO/Tween in PBS | 2064 | 410.31 | 113.85 |

### 1.3.2 Effect of Combination of Vancomycin and aBL in MRSA Biofilm 48 Hours

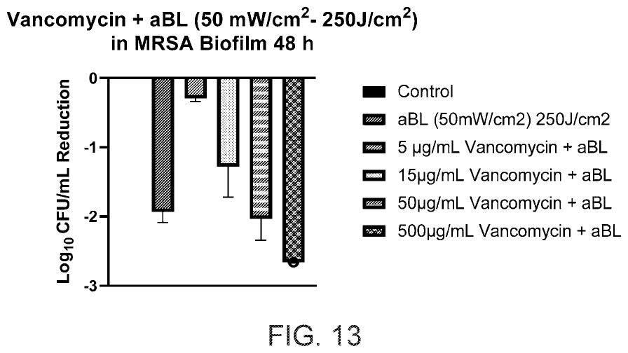

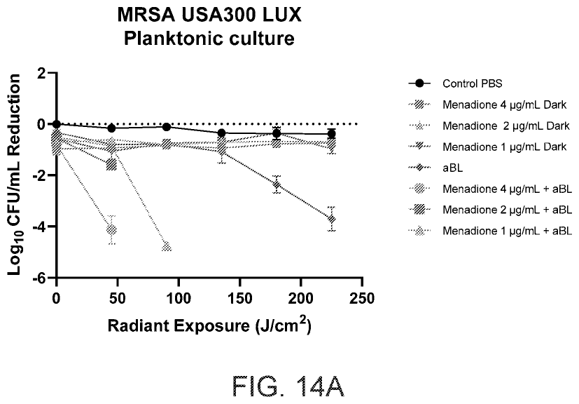

Vancomycin is a common antibiotic that is used to treat serious MRSA skin infectious. We tested different concentrations of vancomycin (5-500 μg/mL) combined with blue light (50 mW/cm²-250 J/cm²) to treat MRSA 48 hours biofilm. Even the higher concentration of vancomycin, 500 μg/mL, which is 25 times above normally safe achievable serum levels, increased killing only by 2.65 log₁₀ CFU/mL in the MRSA biofilm compared to 1.93 log₁₀ CFU/mL of aBL alone. The lower concentrations of vancomycin 5, 15 and 50 μg/mL did not show any improvement of aBL antimicrobial effect (FIG. 13).

### 1.3.3 Minimum Inhibitory Concentrations (MICs) of Menadione in MRSA, P. aeruginosa, and E. coli

Menadione demonstrated antibacterial activity with an MIC of 8 μg/mL, 512 μg/mL, and 1024 μg/mL against MRSA, E. coli, and P. aeruginosa respectively in the absence of blue light or any ambient light. However, we investigated the effects of aBL (50 mW/cm²) in potentiating the antibacterial activities of menadione in the three strains of bacteria. Different fluence values were investigated in different ranges (125 J/cm²-15.62 J/cm²). aBL effectively reduced the MIC values observed when added to menadione. Starting with the lowest fluence value investigated (15.62 J/cm²), the menadione MIC values were found to be 8 μg/mL, 256 μg/mL, and 64 μg/mL for MRSA, E. coli, and P. aeruginosa respectively. It is important to note that aBL is highly effective as an antibacterial since it was able to reduce the MIC of E. coli and P. aeruginosa in as little as 5 minutes irradiating interval. However, the MIC of menadione for MRSA did not change at this fluence rate. Additionally, irradiating the bacteria for 10.5 minutes (31.25 J/cm²) had decreased the menadione MIC values of all bacterial strains to the following values: 4 μg/mL, 256 μg/mL, and 8 μg/mL for MRSA, E. coli, and P. aeruginosa respectively. Furthermore, the fluence of 62.5 J/cm² achieved complete eradication of P. aeruginosa and lowered the MIC values for MRSA and E. coli to 1 μg/mL and 4 μg/ml respectively. This suggests that at even low concentrations, menadione can enhance the microbicidal effectiveness of blue light. The highest fluence that we investigated was 125 J/cm², when we irradiated the bacteria for 42 minutes. Irradiating for this duration at this power density with aBL has completely eradicated all E. coli and P. aeruginosa and lowered the MIC of menadione for MRSA for as low as 0.0625 μg/mL which is 78% lower. Therefore, we observed that the treatment combination of menadione inoculation and aBL irradiation resulted in significantly lower menadione MIC values.

TABLE 2
Minimum Inhibitory Concentration (MIC) of menadione
(vitamin K₃) against bacterial strains in different fluency levels.
Different fluency measures were achieved through
different radiation exposure times.

| Menadione | MRSA | | E. coli | | P. aeruginosa | |
|---|---|---|---|---|---|---|
| 0 J/cm² | 8 | μg/mL | 512 | μg/mL | 1024 | μg/mL |
| (Dark) | | | | | | |
| 125 J/cm² | 0.0625 | μg/mL | — | | — | |
| (42 mins.) | | | | | | |
| 62.5 J/cm² | 1 | μg/mL | 4 | μg/mL | — | |
| (21 mins.) | | | | | | |
| 31.25 J/cm² | 4 | μg/mL | 256 | μg/mL | 8 | μg/mL |
| (10.5 mins.) | | | | | | |
| 15.62 J/cm² | 8 | μg/mL | 256 | μg/mL | 64 | μg/mL |
| (5 mins.) | | | | | | |

### 1.3.4 Menadione Substantially Increases the Antimicrobial Effect of Blue Light in Planktonic Cultures of Bacteria

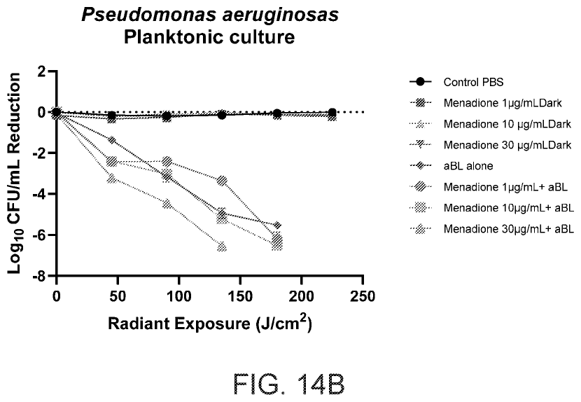

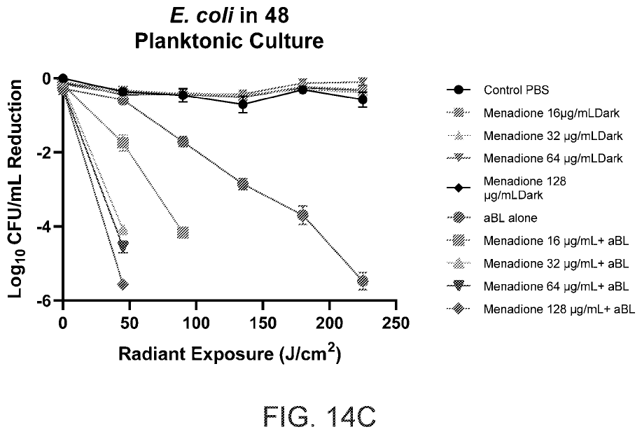

Menadione potentiated the effect of aBL in the planktonic cultures of MRSA, P. aeruginosa, and E. coli. FIGS. 14A-17B show the killing curves obtained by varying the menadione concentration according to the MIC (½ MIC, ¼ MIC, ⅛ MIC, 1/16 MIC) for each strain (MRSA, P. aeruginosa, or E. coli) and exposing the cells to blue light doses at 405 nm (either BL or dark control). The irradiation of aBL alone at 90 J/cm² (50 mW/cm²) inactivated 0.81 log₁₀ CFU/mL of MRSA, 3.08 log₁₀ CFU/mL of P. aeruginosa, and 1.97 log₁₀ CFU/mL of E. coli. However, when the aBL was combined with menadione (⅛ MIC), eradication of 4.59 log₁₀ CFU/mL for MRSA, 4.54 log₁₀ CFU/mL for P. aeruginosa, and 6 log₁₀ for E. coli was observed. Thus, the addition of menadione synergistically enhanced the antimicrobial effects of aBL by approximately-10,000-fold. Also, we observed that the degree of bacterial killing was dependent on the light dose delivered (J/cm²) and the concentration of menadione. It can be seen that P. aeruginosa was substantially more sensitive to aBL compared to the other strains analyzed.

### 1.3.5 Menadione Synergistically Increases the Microbicidal Effect of 405 nm Blue Light on In Vitro Biofilms of MRSA, P. aeruginosa, and E. coli

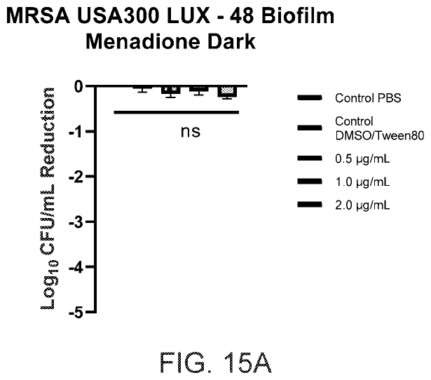

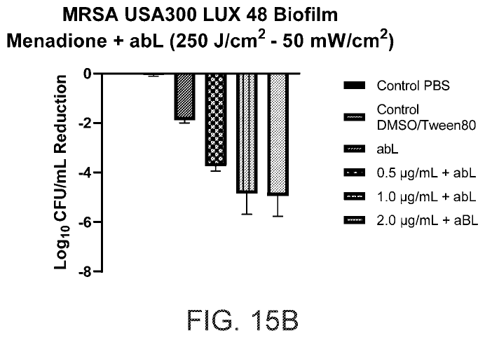

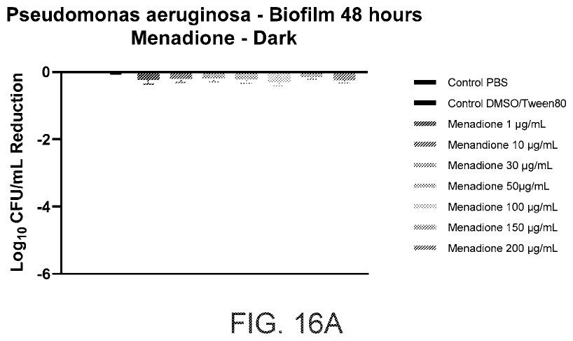

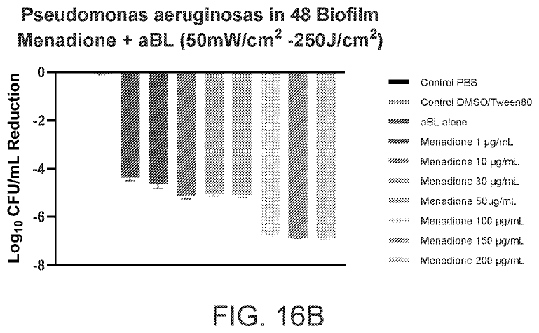

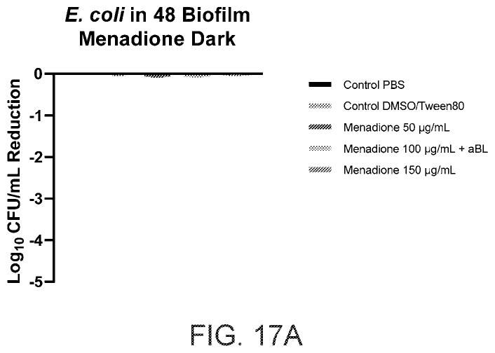

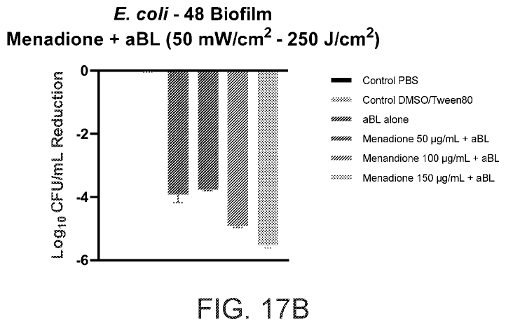

After 48 hours of incubation, mature monomicrobial biofilms of MRSA, P. aeruginosa, and E. coli were obtained with 8.00 log₁₀ CFU/mL per well. After the treatment with the aBL alone at 250 J/cm² (50 mW/cm²), the log₁₀ photoinactivation for each species was 2.14, 4.45, and 3.83 log₁₀ CFU/mL for MRSA, P. aeruginosa and E. coli, respectively. When the biofilms were treated with menadione and 250 J/cm² (50 mW/cm²) aBL combined, all the strains showed increased eradication. In FIG. 15B, 4.85 log₁₀ CFU/mL of MRSA biofilm were killed by 1 μg/mL of Menadione plus aBL, while P. aeruginosa and E. coli required 100 μg/mL to eradicate 6.6 log₁₀ CFU/mL and 4.8 log₁₀ CFU/mL, respectively (see FIGS. 16B and 17B). Therefore, the effect of aBL was synergistically potentiated approximately one thousand-fold when it was combined with menadione in in vitro MRSA biofilms. None of the monomicrobial biofilms showed any significant reduction (<1 log killing) in the presence of menadione without irradiation. P. aeruginosa biofilm was much more sensitive to aBL than the other species, therefore augmentation by menadione was much less.

### 1.3.5.1 Menadione Enables aBL Microbicidal Effects at Lower Power Densities Reducing Potential Adverse Thermal Effects

Temperature was recorded during the aBL irradiation (50 m W/cm²-250 J/cm²-83 minutes) in the 96-well microtiter plates through a thermocouple (OMEGA RDXL4SD). The final temperature measured was 29.9° C., with an increase of only 5° C. from the initial temperature, which did not affect the viability of the microorganisms. Despite the fact that this is below the threshold for pain in human skin, considering the possible intolerance of human tissue to even mild heat, the monomicrobial biofilms of MRSA were also treated with menadione and aBL in lower power densities as 30, 15, and 10 mW/cm². The enhancement of the antimicrobial effect of aBL with menadione was observed even with low power density. At 15 mW/cm² and 250 J/cm² of aBL, 3.01 log₁₀ CFU/mL of MRSA biofilm were killed in the presence of 1 μg/mL of Menadione while the aBL alone in the same conditions was able to inactivate only 1.48 log₁₀ CFU/mL, approximately 102 less (see Table 3).

TABLE 3
Effects of menadione and aBL in MRSA USA 300 biofilm (48 hours
old) at different power densities but similar fluences (doses).

| | | | | | Bacterial Log₁₀ |
|---|---|---|---|---|---|
| | | | | Bacterial Log₁₀ | CFU/mL |
| | | Time Power | | CFU/mL | Reduction after |
| Power Density | Fluence | Density | | reduction after | menadione + |
| (mW/cm²) | (J/cm²) | (mW/cm²) | | aBL | aBL |
| 0 | 250 | 0 | | 0 | 0 |
| 10 | 250 | 6 | hours | −1.19 | −1.91 |
| | | 56 | minutes | | |
| 15 | 250 | 4 | hours | −1.48 | −3.01 |
| | | 37 | minutes | | |
| 30 | 250 | 2 | hours | −1.89 | −2.28 |
| | | 18 | minutes | | |
| 50 | 250 | 1 | hours | −2.16 | −4.85 |
| | | 23 | minutes | | |

### 1.3.6 Photoinactivation of Bacterial Biofilms in Ex-Vivo Porcine Partial Thickness Wounds by Menadione+Antimicrobial Blue Light

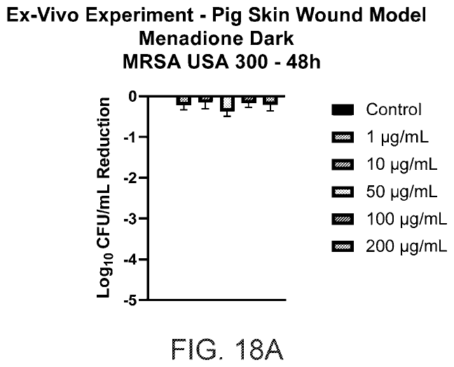

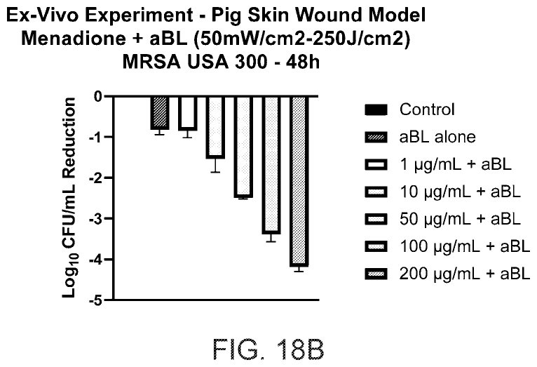

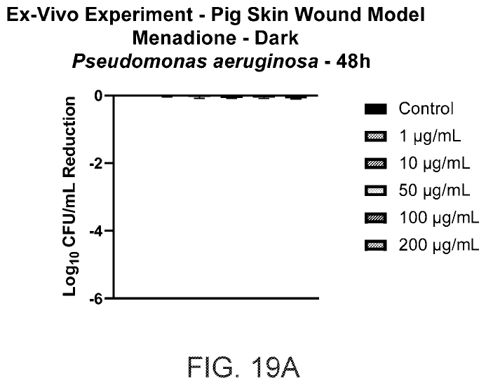

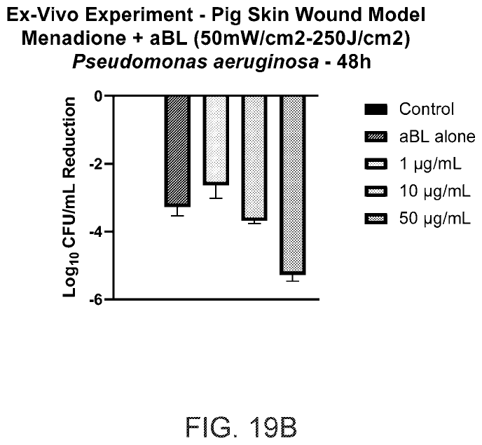

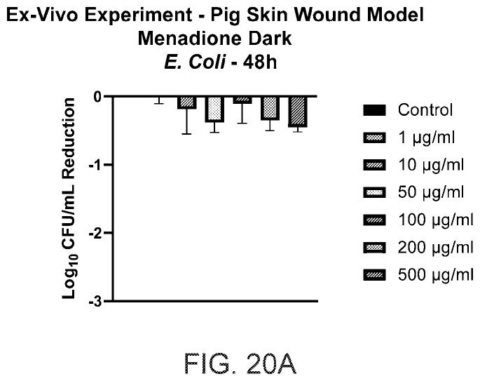

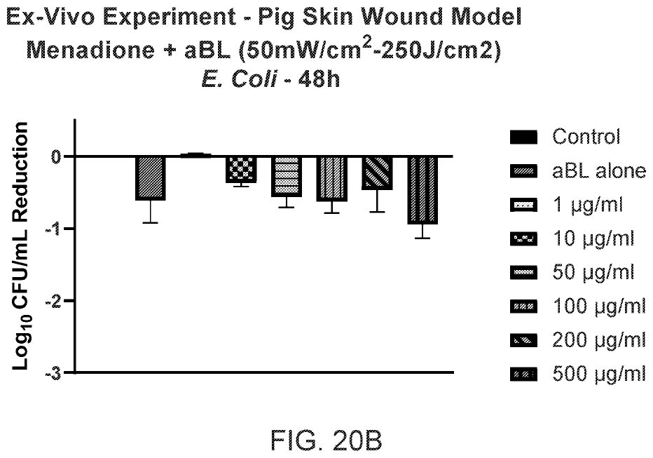

48 hours following the inoculation, mature monobacterial biofilms of MRSA, P. aeruginosa, and E. coli were obtained with 8.00 log₁₀ CFU/g per wound bed on each explant. MRSA biofilms in explant were reduced by 0.81 log₁₀ CFU/g after the treatment of just aBL 250 J/cm² (50 mW/cm²). However, MRSA biofilms in ex-vivo porcine skin were reduced by up to 4.18 log₁₀ CFU/g when treated with aBL combined with menadione (200 μg/mL). For MRSA biofilms, the log reduction of mature monobacterial biofilm increased as the menadione concentration increased (see FIGS. 16A-16B). Another bacterium we investigated in porcine explants was P. aeruginosa, for which we observed 3.27 log₁₀ CFU/mL reduction when treated with blue light alone, while further reduced by 5.27 log₁₀ CFU/mL with a with combination of 50 μg/mL of menadione and blue light. For MRSA and P. aeruginosa biofilms in ex-vivo explants, the addition of menadione potentiated aBL antibacterial effects. For both of these bacterial strains, biofilms that were treated with menadione in the absence of aBL radiation produced no effect of biofilm reduction. E. coli biofilms in ex-vivo explants did not respond to the treatment of aBL and Menadione. See FIGS. 18A-20B.

### 1.3.7 Investigating the Ability of Menadione to Enhance ROS Levels by aBL and Potential Function as a Photosensitizer

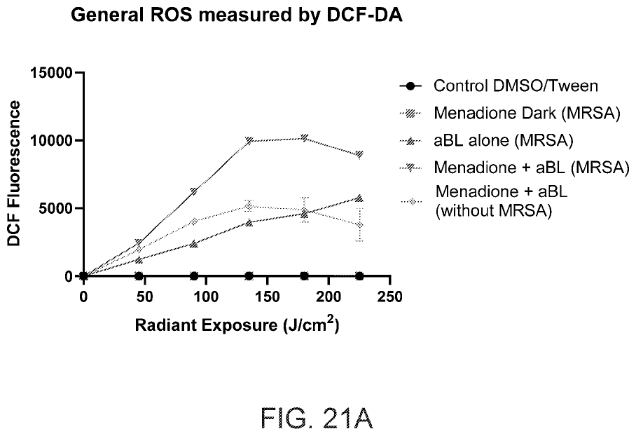

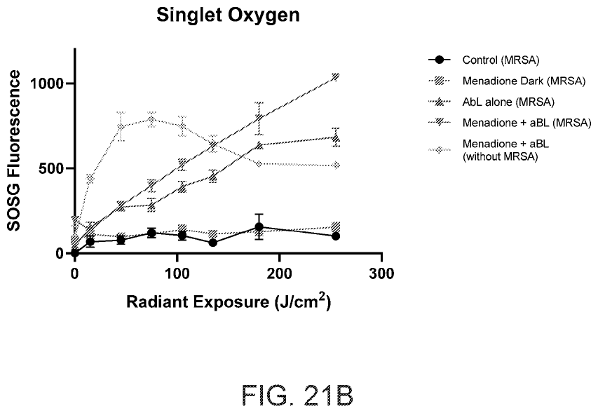

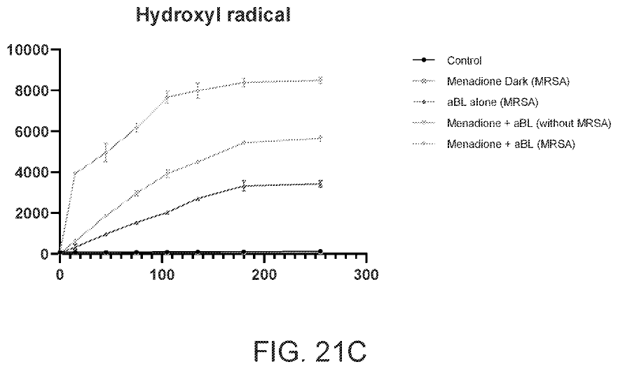

Observing that there was some, albeit minimal, absorption of 405 nm light by menadione, and that together they synergistically increased bactericidal activity, we investigated possibility that menadione might be acting as a photosensitizer. To do so, the production of ROS by menadione combined with blue light was measured by different ROS probes, DCF-DA, SOSG and HPF, in the presence and absence of MRSA. The acetylated forms of 2′,7′-dichlorofluorescein (DCF-DA) is nonfluorescent until the acetate groups are removed by intracellular esterase and oxidation occurs within the cell. Oxidation of these probes can be detected by monitoring the increasing of fluorescence signals. The SOSG reagent is highly selective for 102 while the HPF is selective for OH. Both do not fluoresce until they react with their specific substrate, which means the signal of fluorescence is be related to specific ROS production. In FIGS. 21A-21C, in the absence of bacteria, we can see the probes generated higher fluorescence signal for all radicals where menadione was exposed to the blue light, while the control and menadione dark group signals remained negligible. This observation illustrated that menadione when combined with blue light at 405 nm produced general ROS, 102, and OH. This suggests that menadione/vitamin K₃ may be potentially acting as a photosensitizer under blue light irradiation (405 nm), and that reactions type I and II are potentially taking place intracellularly.

The general ROS production also was tested in presence or absence of MRSA to evaluate whether menadione was directing with the bacteria to potentiate the aBL effect or not. The result presented in FIGS. 21A-21C shows that aBL increased fluorescence signal of all probes as long as the bacteria were exposed to the blue light. When the bacteria were incubated with menadione and irradiated to 405 nm, the fluorescence of all probes were higher than the aBL alone. The production of ROS measured by DCF was two times higher in the presence of menadione+aBL than the aBL alone (180 J/cm²), while singlet oxygen showed 1.5 (200 J/cm²) times higher and hydroxyl radical 1.77 times higher (135 J/cm²).

### 1.4 Discussion

Our studies demonstrate the efficacy of Vitamin K₃, menadione, to synergistically potentiate the antimicrobial effect of 405 nm blue light against several critical human multi-drug resistant (MDR) pathogenic bacteria. Antimicrobial resistance (AMR) has become a major public health threat worldwide [Ref. 4]. Despite current efforts, treatment options for MDR bacteria remain limited, and are only becoming more so as bacteria become increasingly resistant. The effect of aBL and its potential for multi-log killing of bacteria in bacterial planktonic cultures in different bacterial species, as well as many fungi, is well documented in the literature [Ref. 8].

It is interesting to note that in the recent comprehensive review by Cook and Wright [Ref. 14] in which alternatives to classical antibiotics are reviewed, antimicrobial blue light is not even mentioned in passing. It is in that context that we have sought to examine the effectiveness of blue light therapy of biofilm infections with the intent of ultimately translating this to clinical use when feasible, and specifically whether or not we could overcome some of the resistance to all types of therapy that biofilms represent by the use of an adjuvant therapy to increase the ROS formation that is the basis of phototherapy of infection.

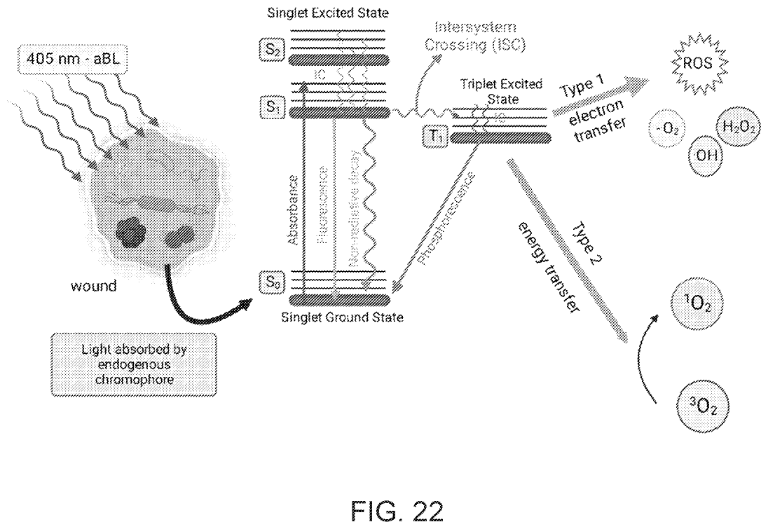

While the mechanism of action of aBL is still not fully understood, the prevailing hypothesis is that endogenous chromophores in bacteria are able to absorb blue light and promote photochemical reactions. These photochemical reactions can be explained based on Jablonski Diagram in FIG. 22. The endogenous chromophore absorbs photons of light with specific energy raising the electrons from the ground state (S₀) to the singlet-excited state (S₁). This photon-induced higher-energy orbital S₁ is very unstable and can lose the energy for radiative decay (fluorescence) or non-radiative decay (heat) to come back to the S₀. However, the electrons in the excited singlet S₁ state of the endogenous chromophore may enter a more stable excited triplet state (T₁), through a process known as “intersystem crossing”. In T₁, the electrons can come back to the S₀ by emitting a phosphorescent photon or depending on the lifetime of the triplet state, can drive the pathway for Type I or Type II photochemical process, or both concomitantly. A Type I reaction involves electron transfer reactions with acquisitions or donations of electrons, which forms reactive oxygen species (ROS), as superoxide anion (O₂′⁻), hydrogen peroxide (H₂O₂) and hydroxyl radicals (HO*). In the Type II reaction, there occurs an energy transfer between the triplet state of the endogenous chromophore and the molecular oxygen, leading to the formation of singlet oxygen (¹O₂). Both pathways are thus capable of generating ROS, with the resulting potential downstream effects [Ref. 23]. Thus, it is not only the light energy source that is critical, but so is the endogenous or exogenous chromophore. Based on these concepts, it is clear that factors affecting light absorption will have a major impact on the resulting light/target interactions.

In that context, bacterial wound biofilms are enclosed in an extracellular polymeric matrix, in which the relatively impermeable and often opaque matrix may partially explain their resistance to aBL eradication. Furthermore, in bacterial biofilms, bacteria tend to go into a metabolically dormant state, forming what are termed “persisters”, reducing their sensitivity to antibiotics, but also potentially to photo-reactivity. [Ref. 31]. For example, in our 48-hour in vitro biofilm model, vancomycin, a common antibiotic that is often used to treat serious MRSA skin infections, had only minimal effects on MRSA biofilms when combined with aBL and vancomycin was applied at an extraordinary concentration (1 mg/mL), which is 50 times above normally achievable serum levels (see FIG. 13).

For many years, mean bactericidal concentration (MBC) has been defined as 99.9% killing of organisms, or 3 logs of killing [Ref. 24]. Both with antibiotics and with aBL, bacteria in biofilms have proven extraordinarily (100-10,000× more) resistant, and this level of killing has proven especially difficult with MRSA in biofilms. Ferrer-Espada et al. [Ref. 26] studied aBL in 48 hour monomicrobial and polymicrobial biofilms and found that a fluence of 216 J/cm² of aBL (60 mW/cm²) was able to eradicate just 1.62 log₁₀ CFU/mL of MRSA USA300 and 3.67 log₁₀ CFU/mL of Pseudomonas aeruginosa.

Similarly, our findings demonstrate that aBL alone was not effective in eliminating more than 2 logs in MRSA biofilms in vitro, nor in the ex-vivo skin wounds. Therefore, we focused on exploring a strategy that enhances aBL activity through combining it with topical vitamin K₃ (menadione) to increase the endogenous bacterial ROS production. Although there have been several studies representing the combination of aBL and adjuvants for MDR bacteria treatments [Ref. 17], this study represents the first that identifies Vitamin K₃ (menadione) as a clinically viable strategy to potentiate aBL effectiveness in the killing of MDR bacteria, including MDR bacteria in biofilms, a major pathogenic mechanism in bacterial infections.

The synergistic effect on blue light ROS production was demonstrated when menadione was applied to the bacteria combined with aBL, showing a 2.5-fold increase of total ROS (see FIG. 21). The amounts of reactive oxygen species, such as hydroxyl radical, hydrogen peroxide, singlet oxygen and anion superoxide generated by 10 μM menadione and aBL were significantly greater than the blue light alone (FIG. 21). However, we did not observe the same levels of oxygen radicals with menadione alone in the absence of light, confirming that the mechanism of action of menadione when combined with blue light is to contribute to the photoreactions of type I and type II from aBL to enhance oxidative stress, rather than to initiate it. It can be fairly stated that our blue light LED generates a radiant spectrum from 380-410 nm, with a sharp peak at 405 nm. That would generate some light in the UV-A part of the spectrum, where menadione has a peak absorption, but menadione also shows absorption with the tail all the way to 410 nm (see FIG. 12B).

It has been reported that menadione and some other quinones have analogous effects in generating reactive oxygen species through the catalytic reduction of oxygen with electrons that would have otherwise been involved in the electron transport chain in cellular respiration [Ref. 9]. The redox reaction generates reactive oxygen species through the reduction of oxygen and these ROS can induce oxidative damage to the bacteria. Schlievert et al. [Ref. 18] compared the ability of menadione to inhibit growth of S. aureus MN8 under anaerobic and aerobic conditions and found menadione was 4-fold more bactericidal for S. aureus when cultured aerobically. In the study, the author showed that the increased antimicrobial effect in the presence of O₂ presumably reflects the important role that menadione plays as an inducer of redox cycling. In addition, another known effect of the blue light is a stimulation of electron transfer and proton motive force, that consequently increases both ROS and ATP production [Ref. 30] in bacteria. Without intending to be bound by theory, we hypothesize that the redox cycling activity of menadione is greatly augmented by the ROS generated by blue light. We further conclude that the ROS produced by menadione, combined with ROS generated by aBL, significantly increases bacterial intracellular oxidative stress, resulting in antimicrobial synergism.

We found an enhancement up to 104-fold in antimicrobial effects when the aBL was combined with menadione in MRSA and E. coli planktonic culture. Our findings further demonstrated that menadione synergistically potentiated the blue light antimicrobial activity even in the microbial biofilms (48 hours old). The effects of aBL were potentiated approximately 1000-fold when it was combined with menadione in MRSA and 100-fold in P. aeruginosa biofilms. The increase in effectiveness was also observed even with low power density of blue light in MRSA biofilms, an advantage in terms of tolerability in human tissue, heat dissipation and future clinical applicability to infected wounds. E. coli biofilms showed more resistance to treatment with the combination, but even these showed an enhancement of 1 log-fold killing in comparison with aBL alone.

We also studied the effects of menadione and aBL in bacterial biofilms in an ex vivo porcine wound model. The advantage of using a porcine ex vivo wound model is the similarity to human skin, enabling us to study a biofilm adhered to the skin, with high reproducibility and reasonable cost [Ref. 30]. An important finding was the significant potentiation of the antimicrobial effects by menadione when added to aBL in the ex vivo porcine wound model, which we are further investigating as a practical and inexpensive stimulant for screening candidate combination therapies for treating human skin wound biofilm infections with various forms of the light therapy. We found that the addition of low doses of topical menadione to aBL in the ex vivo model improved the aBL activity in MRSA by 104-fold and in P. aeruginosa 102-fold in comparison to aBL alone, consistent with our earlier in vitro biofilm treatment results, but now in a more challenging model.

Our findings demonstrate a significant potentiation of the antimicrobial effects of aBL with menadione in planktonic culture, biofilms and the ex vivo porcine wound model. Menadione alone did not kill bacteria in either the 48-hour biofilms in vitro or the ex vivo wound model, while showing some minimal antimicrobial activity against pathogens in planktonic culture as a single agent in the dark.

Menadione may produce other toxic effects to the bacterium besides direct oxidative damage. According to Andrade et al. [Ref. 8] due to its lipophilic character, menadione can interfere with bacterial membrane integrity, resulting in changes in membrane morphology and physiology and an increase of bacterial membrane permeability. In their study, the authors showed menadione led to an increase in aminoglycoside sensitivity by a membrane permeability mechanism in Staphylococcus aureus, but not against E. coli or Pseudomonas aeruginosa. In addition, Tintino et al. [Ref. 9] demonstrated inhibition of the norA gene by menadione. This is a critical antimicrobial resistance gene controlling the NorA efflux pump, and they demonstrated significant inhibition of both efflux pump gene expression and efflux pump function, restoring some degree of norfloxacin (fluoroquinolone) sensitivity, a potentially important clinically-applicable finding for treating surface wounds reachable by light and/or menadione.

In the absolute dark, menadione does not appear to generate significant ROS in bacteria. In bacteria exposed to blue light, menadione results in a substantial increase in ROS production by bacteria, by acting, our findings imply, as an exogenous photosensitizer to absorb photons and generate ROS as well acting as an ROS recycler. Combined with endogenous bacterial chromophores absorbing photons of antimicrobial blue light to generate endogenous ROS, which menadione regenerates, this results in a potentially toxic load of ROS to the bacteria, causing damage to bacterial membranes, efflux pumps, and bacterial DNA. The redundant antioxidant systems of mammalian cells are better protected, but as this therapy is advanced, this will have to be carefully examined.

Interestingly, Zheng and colleagues [Ref. 25] have demonstrated that menadione can act as a selective, potent inhibitor of the NLRP3 inflammasome, a multi-protein complex the activation of which, in humans, results in intense inflammatory reactions such as gout. The simultaneous improvement of antimicrobial activity with the potential for some local anti-inflammatory activity could prove quite beneficial in the topical treatment of wounds clinically.

### 1.5 Conclusions

In conclusion, we have demonstrated that the addition of menadione synergistically potentiated the effects of aBL, increasing bacterial oxidative stress to significantly increase microbicidal killing in a synergistic pattern in MRSA and P. aeruginosa, while having less effect on E. coli, less endowed with endogenous chromophores. In addition to enhancing ROS production by aBL, menadione has other effects, such as stimulating electron transport, inhibition of critical bacterial efflux pumps, and increased permeability of the bacterial membrane.

Our findings teach that the combination of aBL and menadione can be an effective alternative clinical strategy for the treatment of antimicrobial drug-resistant (AMR) bacteria-infected wounds capable of being reached by antimicrobial blue light. This is especially applicable to chronic wounds infected by AMR bacteria by potentially avoiding the use of antibiotics for the treatment of biofilm infections that often require prolonged courses of antimicrobial therapy. These pre-translational studies suggest that menadione can further amplify the utility of a “topical” therapy, antimicrobial blue light, with the potential to avoid further development of antimicrobial resistance, while providing synergistic “log-killing” of AMR bacteria in biofilm infections.

In conclusion, we have demonstrated that the addition of menadione synergistically potentiated the effects of aBL, increasing bacterial oxidative stress to significantly increase microbicidal killing in a synergistic pattern in MRSA and Pseudomonas aeruginosa, while having less effect on E. coli, less endowed with endogenous chromophores. In addition to enhancing ROS production by aBL, menadione has other effects, such as stimulating electron transport, inhibition of critical bacterial efflux pumps, and increased permeability of the bacterial membrane. We have shown that antimicrobial adjuvants (e.g., menadione) can be used in conjunction with antimicrobial blue light to reduce the bacterial bioburden in biofilms.

### REFERENCES FOR EXAMPLE 1

1. Hernando-Amado et al., “Defining and combating antibiotic resistance from One Health and Global Health perspectives”, Nature Microbiology. 2019; 4:1432-1442.
2. Stracy et al. “Minimizing treatment-induced emergence of antibiotic resistance in bacterial infections”, Science (New York, N.Y.). 2022; 375:889-894.
3. Rice et al., “Burden of Diabetic Foot Ulcers for Medicare and Private Insurers”, Diabetes Care. 2014; 37:651.
4. Lipsky et al., Infectious Diseases Society of America clinical practice guideline for the diagnosis and treatment of diabetic foot infections. Clin Infect Dis. 2012; 54: e132-173.
5. Akhi et al., “Frequency of MRSA in diabetic foot infections”, International Journal of Diabetes in Developing Countries. 2017; 37:58-62.
6. Wang et al., “Antimicrobial blue light inactivation of pathogenic microbes: State of the art”, Drug Resistance Updates. 2017; 33:1-22.
7. Truong et al., “Emerging Issues in Vitamin K Research”, Journal of Evidence-Based Complementary & Alternative Medicine. 2011; 16:73-79.
8. Andrade et al., “Menadione (vitamin K) enhances the antibiotic activity of drugs by cell membrane permeabilization mechanism”, Saudi J Biol Sci. 2017; 24:59-64.
9. Tintino et al., “Effect of Vitamin K3 Inhibiting the Function of NorA Efflux Pump and Its Gene Expression on Staphylococcus aureus”, Membranes (Basel) 2020; 10.
10. Tang et al., “Synthesis of mitochondria-targeted menadione cation derivatives: Inhibiting mitochondrial thioredoxin reductase (TrxR2) and inducing apoptosis in MGC-803 cells”, Bioorg Med Chem Lett. 2022; 60:128586.
11. Beck et al., “Menadione reduction by pharmacological doses of ascorbate induces an oxidative stress that kills breast cancer cells”, Int J Toxicol. 2009; 28:33-42.
12. Klotz et al., “1, 4-naphthoquinones: from oxidative damage to cellular and inter-cellular signaling”, Molecules. 2014; 19 (9): 14902-14918.
13. Murray et al., “Global burden of bacterial antimicrobial resistance in 2019: a systematic analysis”, The Lancet. 2022; 399 (10325): 629-655.
14. Cook et al., “The past, present, and future of antibiotics”, Science Translational Medicine. 2022; 14 (657).
15. Percival et al., “A review of the scientific evidence for biofilms in wounds”, Wound Repair and Regeneration. 2012 Sep. 1, 2012; 20 (5): 647-657.
16. Banerjee et al., “A review on basic biology of bacterial biofilm infections and their treatments by nanotechnology-based approaches”, Proceedings of the National Academy of Sciences, India Section B: Biological Sciences. 2020; 90 (2): 243-259.
17. Leanse et al., “Antimicrobial blue light: A ‘Magic Bullet’ for the 21st century and beyond?”, Advanced Drug Delivery Reviews 2022; 180:114057.
18. Schlievert et al., “Menaquinone analogs inhibit growth of bacterial pathogens”, Antimicrobial Agents and Chemotherapy. November 2013; 57 (11): 5432-7.
19. Hassan et al., “Menadione”, Profiles of drug substances, excipients and related methodology 2013; 38:227-313.
20. Vidlak et al., “Infectious dose dictates the host response during Staphylococcus aureus orthopedic-implant biofilm infection”, Infection and immunity. 2016; 84 (7): 1957-1965.
21. Jett et al., “Simplified agar plate method for quantifying viable bacteria”, Biotechniques. 1997; 23 (4): 648-650.
22. Probes M. Invitrogen detection technologies. Product Information. 2004 2004; (Reactive Oxygen Species (ROS) Detection Reagents): 1-5.
23. Abrahamse et al., “New photosensitizers for photodynamic therapy”, Biochemical Journal. 2016; 473 (4): 347-364.
24. Reller et al., “Serum Dilution Test for Bactericidal Activity. II. Standardization and Correlation with Antimicrobial Assays and Susceptibility Tests”, The Journal of Infectious Diseases, Volume 136, Issue 2, 136 196-204 (1977).
25. Zheng et al., “Synthetic vitamin K analogs inhibit inflammation by targeting the NLRP3 inflammasome”, Cellular & Molecular Immunology. 2021 Oct. 1 2021; 18 (10): 2422-2430.
26. Ferrer-Espada et al., “Antimicrobial Blue Light Inactivation of Microbial Isolates in Biofilms”, Lasers in Surgery and Medicine, Volume 52, Issue 5, 2020.
27. Ferrer-Espada et al., “Antimicrobial Blue Light Inactivation of Polymicrobial Biofilms”, Frontiers in Microbiology, April 2019.
28. Haug et al., “The impact of the length of total and intravenous systemic antibiotic therapy for the remission of diabetic foot infections”, International Journal of Infectious Diseases, Volume 120, July 2022, Pages 179-186.
29. Seaton et al., “Porcine Models of Cutaneous Wound Healing”, ILAR Journal, Volume 56, Issue 1, 2015, Pages 127-138.
30. Yang et al., “Development of a novel ex vivo porcine skin explant model for the assessment of mature bacterial biofilms”, Wound Repair and Regeneration Volume 21, Issue 5, 2013.
31. Brynildsen et al., “Potentiating antibacterial activity by predictably enhancing endogenous microbial ROS production”, Nat Biotechnol. 2013 February; 31 (2): 160-165.
32. Schlievert et al., “Menaquinone Analogs Inhibit Growth of Bacterial Pathogens”, Antimicrob Agents Chemother. 2013 November 57 (11): 5432-5437.

The citation of any document or reference is not to be construed as an admission that it is prior art with respect to the present invention.

### Example 2

We developed a prototype antimicrobial blue light bandage that is low power, potentially battery operated and thus portable, and capable of providing microbial suppression and microbial killing for prolonged periods of time using battery power in the field. Our device was made of inexpensive components that are easily scaled for mass manufacture. The device: (1) uses inexpensive LEDs at low power; (2) is low power, thus enabling battery-powered; (3) is specifically designed for low cost, mass manufacture; and (4) is light weight for use in the field by military, ERs, ambulances etc.

Existing U.S. military guidelines for the prevention of infections associated with combat related injuries include bandaging with a sterile dressing and transportation to a higher level of care. Administration of antibiotics at the point of care is advised according to the guidelines, especially if evacuation is delayed. It is anticipated that in the future, evacuation will become increasingly problematic. There is thus a need to stabilize and maintain a reduced bacterial load in wounds in the field for prolonged periods during which time continued antibiotic administration may not be optimal. We thus developed a prototype wound dressing capable of delivering prolonged antimicrobial activity, for one or more hours, days, weeks, months, or years. Specifically, we applied the use of antimicrobial blue light (aBL), which has shown promise as a nonantibiotic approach to combat microbial infections. ABL uses irradiation with visible light in a wavelength range of 400-470 nm and the production of reactive oxygen species (ROS), highly cytotoxic against bacteria. Phototherapy has been demonstrated to have potent antimicrobial properties against a broad range of microbes, including gram-positive and negative bacteria, mycobacteria, fungi and biofilms, demonstrated in in vitro, animal studies and at least one clinical trial. The advantages of local aBL treatment, compared to systemic antibiotics, include highly specific local targeting of bacterial burden, reduced risk of developing antimicrobial multi-drug resistant (MDR) infections, and avoidance of systemic side effects.

### Methods

We first established desired features for a dressing in some use environments: lightweight and portable; low power for battery operation; easily mass manufactured from inexpensive components; biocompatible; light-transmitting; non-fouling; and highly bactericidal. The device may be used for initial treatment of a wound to prevent the development of a high bacterial wound burden and/or as treatment for an already contaminated wound. Inexpensive blue light (e.g., 405 nm) LEDs are located in a covering above the bandage, which sits in contact with the wound. We replicated this design for initial in vitro testing of bactericidal activity, varying power density, time of exposure, and thus radiant exposure. Bacterial viability was performed in planktonic and biofilm cultures of methicillin resistant Staphylococcus aureus (MRSA), methicillin-susceptible Staphylococcus aureus (MSSA), and Pseudomonas aeruginosa using aBL transmitted through the prototype bandage. Bacteria were quantified by counting colony-forming units/mL (CFU/mL). Different power densities (15, 30, 50 and 60 mW/cm²), time exposures and thus radiant exposures (radiant exposure=power density×time in seconds) of aBL were evaluated (0-720 J/cm²). Bactericidal activity was quantitated by reduction of CFU/mL of viable bacteria in both planktonic culture and in biofilms. Staphylococcus aureus suspensions containing 10⁸ CFU/mL were exposed to continuous-wave 405 nm light for different power densities and radiant exposures. The monomicrobial biofilm was grown by inoculating 106 CFU/mL for MSSA, MRSA and Pseudomonas aeruginosa in 96-well microtiter plates. An issue using phototherapy is potential overheating on the skin caused by long periods of light exposure and power consumption for a portable device. To address these issues, we additionally studied the effects of varying the illumination duty cycle (50%; 10 seconds time on and 10 seconds off; or continuous-wave, 100%).

### Results

For MSSA planktonic culture, following 432 J/cm², with continuous-wave 405 nm light through the bandage at power densities of 60, 30 and 15 mW/cm², bactericidal activity was log reduced by 6.09, 4.67 and 2.84 CFU/mL, respectively. MSSA and MRSA were exposed to 405 nm light, duty cycle 50% at 50 mW/cm², radiant exposure 360 J/cm², resulting in log reduction of 5.05 for MSSA and 4.43 CFU/mL for MRSA. Temperature inside the target microtiter wells after continuous-wave 8-hour blue light treatment was 38.2° C., while the maximum temperature reached 27.8° C. after 24-hour with 50% duty cycle. We investigated 405-nm ABL inactivation of biofilms using the prototype device with 50% duty cycle. For 24-hour old monomicrobial biofilms formed in 96-well microtiter plates, 3.28-log CFU inactivation of MSSA and 1.87-log CFU inactivation of MRSA were observed after an ABL exposure of 720 J/cm² through the bandage, at 50 mW/cm², with 8-hour exposure time. However, when power density was decreased to 15 mW/cm² with an increase of exposure time to 24 hours, (radiant exposure 648 J/cm²) the 24-hour old MRSA biofilm underwent a 2.72-log CFU inactivation while Pseudomonas aeruginosa underwent 5.77-log inactivation.

### Conclusions

We developed a prototype antimicrobial blue light bandage that is low power, potentially battery operated and thus portable, and capable of providing microbial suppression and microbial killing for prolonged periods of time using battery power in the field. Our device is made of components that are easily scaled for inexpensive manufacture. We tested our prototype device by measuring in vitro killing of MSSA, MRSA, and Pseudomonas aeruginosa in both planktonic and biofilm cultures. We measured the effects of varying light radiation dosimetry parameters, including power density (mW/cm²) and radiant exposure (J/cm²), as well as duty cycle. The dissipation of heat is a major advantage of a duty cycle over continuous wave, particularly for a burn application. When the prototype aBL bandage was tested in vitro against planktonic MSSA, MSRA, and Pseudomonas aeruginosa approximately 3 logs or more of bactericidal activity are achieved. Similar bactericidal activity can be achieved with bacterial biofilms at low power densities using prolonged exposure times, dissipating heat with a duty cycle, resulting in high radiant exposures.

### Example 3

A prototype wound dressing capable of delivering prolonged antimicrobial activity by the dressing for as long as 7 days was developed. Specifically, we sought to apply the use of antimicrobial blue light as a non-antibiotic approach to reducing the bacterial burden in combat wounds. Antimicrobial blue light within the range of 400-470 nm has been demonstrated to produce reactive oxygen species that are highly cytotoxic against a broad range of microbes, including gram-positive and gram-negative bacteria, fungi, mycobacteria and biofilms containing these organisms. The advantages of local antimicrobial blue light treatment, compared to systemic antibiotics, include high specificity for microbes with correspondingly low cytotoxicity to mammalian cells, reduced risk of developing antimicrobial multidrug resistant (MDR) infections, and avoidance of systemic side effects seen with antibiotics. However, there was no available, portable antimicrobial blue light wound dressing for field use.

To address this military need, we have developed a prototype surface wound dressing, which we have termed the Photonic Antimicrobial Wound Surface Dressing (PAWS-Dressing), and evaluated and refined the prototype.

We first established criteria for an example embodiment or configuration of such a dressing: biocompatible; light-transmitting; highly bactericidal; non-fouling; lightweight and portable; low power for battery operation; made of inexpensive components for mass manufacture and disposability. We envisioned using the device for initial treatment of a wound to prevent the development of a high bacterial wound burden and/or as treatment for an already contaminated wound.

Based on this, we developed a prototype device using a 3-D printer and cast polydimethylsiloxane (PDMS) to be used as a dressing. PDMS is an FDA approved for use in disposable soft contact lenses, and as such is highly surface biocompatible, hydrophobic, oxygen permeable, breathable, highly translucent, flexible and moldable to fit the desired target area. PDMS is also inexpensive. Blue light (405 nm) LEDs were located above the PDMS bandage in a silicon LED-holder.

Immediately beneath the LEDs were inexpensive plastic convex lenses which uniformly distribute the light to the wound site. The PDMS bandage sits in contact with the wound, suspended off the wound by, in this example embodiment, about 2-8 millimeters (e.g., about 5 millimeters) on flexible PDMS “feet”, which enable gentle suction of exudate from the wound by an inexpensive Jackson-Pratt-like suction drain. We explored a number of patterns to optimize fluid flow and uniform light distribution. A wafer-thin, solid-state controller connected to a rechargeable portable battery sits on top of the LED holder. A clear Tegaderm-like adhesive tape holds the PDMS bandage on the wound, enabling gentle suction of exudate if present; the silicon LED holder can be secured to the surrounding uninjured skin with a standard adhesive bandage. Other issues when using phototherapy are potential overheating on the skin caused by long periods of light exposure and power consumption for a portable device. To address these issues, we additionally studied the effects of varying the illumination duty cycle (50% or continuous-wave; 10 seconds on and 10 seconds off).

### Example 4

We investigated other ways to enhance antimicrobial blue light to make it more widely useable against bacterial biofilms.

A bacterial suspension was grown in a shaking incubator overnight in BHI broth. Cells were collected by centrifugation at 4,000 rpm for 5 minutes and suspended in PBS at a density of 106 CFU/mL. Then, the biofilms were grown for 48 hours in 96-well microtiter plates with a renewal of the media every 24 hours. Following the 48-hour biofilm growth, fresh 200 μL of PBS with the tetracyclines minocycline, doxycycline, and demeclocycline in different concentrations were added to the wells and the biofilms were incubated in the dark at 25° C. for 1 hour: 0, 0.25, 0.5, and 1 μg/mL for minocycline; 0, 0.5, 1, and 2 μg/mL for doxycycline; and 0, 0.5, 1, and 2 for demeclocycline. After that, the control and the tetracycline-treated biofilms were maintained without irradiation (“menadione/dark”). Simultaneously, the aBL (405 nm) was delivered to the other biofilm groups testing different power densities (50, 30 mW/cm²) while keeping the radiant exposures of 50-250 J/cm². After the light exposure, the biofilms were carefully washed two times with PBS and 200 μL of PBS was added to each well. Then, the bacterial biofilms in each well were harvested by scratching with a sterile pipette tip and three wells of each group were pooled together in a 1.5 mL microcentrifuge tube. The total volume obtained, 600 μL (3 wells), was sonicated using a Branson 2510 Water Bath Sonicator (Marshall Scientific, LLC) for 5 minutes and the CFU/mL was determined by 10-fold serial dilutions in PBS on BHI agar plates. CFU was counted after overnight incubation at 37° C.

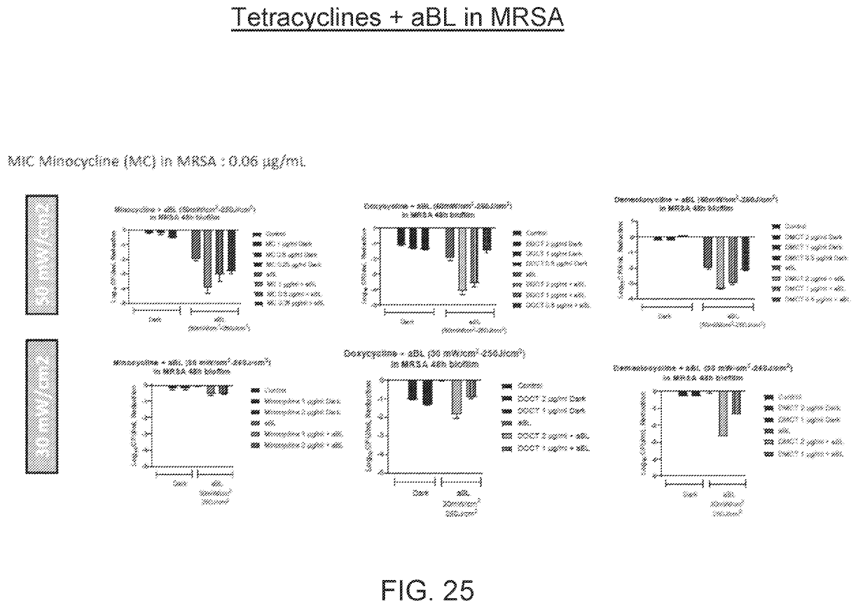

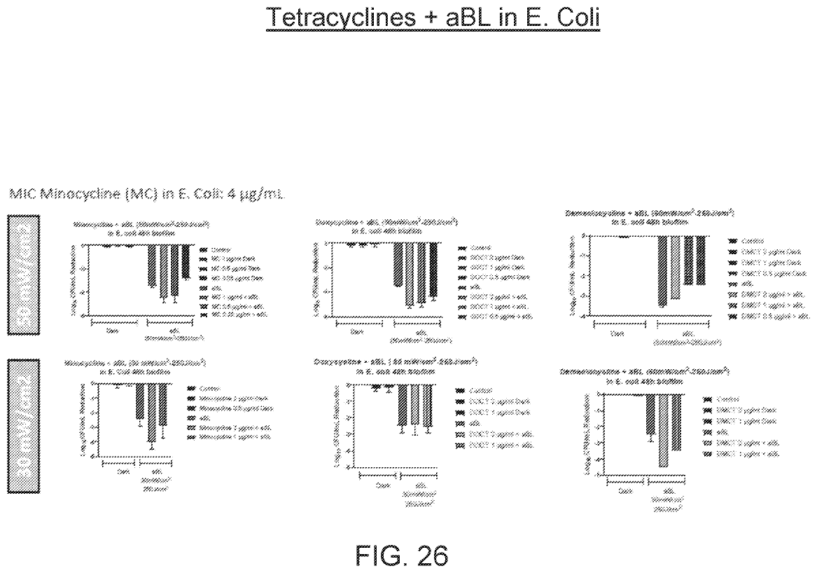

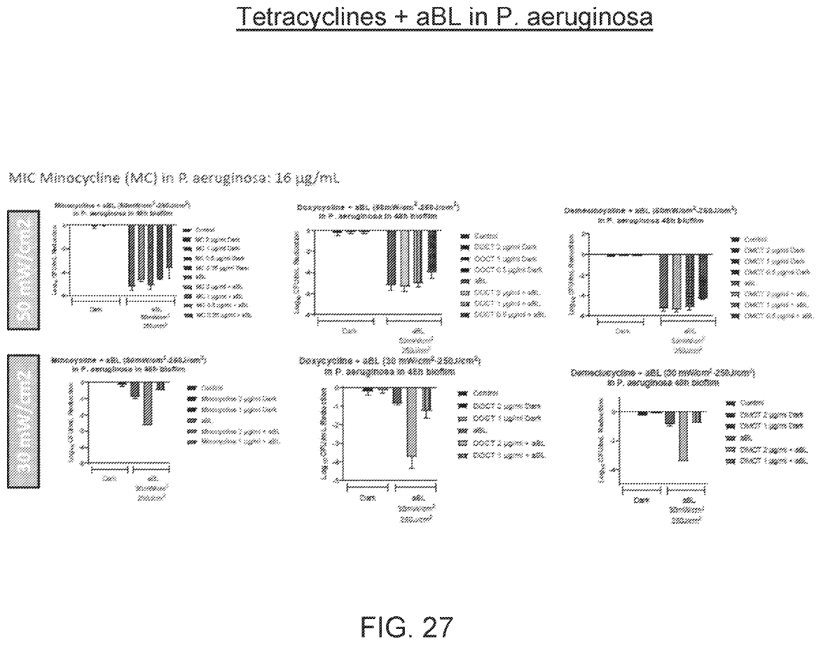

We found an enhancement in antimicrobial effects when the aBL was combined with minocycline, doxycycline, or demeclocycline in MRSA biofilms (see FIG. 25). We found an enhancement in antimicrobial effects when the aBL was combined with minocycline, doxycycline, or demeclocycline in E. coli biofilms (see FIG. 26). We found an enhancement in antimicrobial effects when the aBL was combined with minocycline, doxycycline, or demeclocycline in P. aeruginosa biofilms (see FIG. 27).

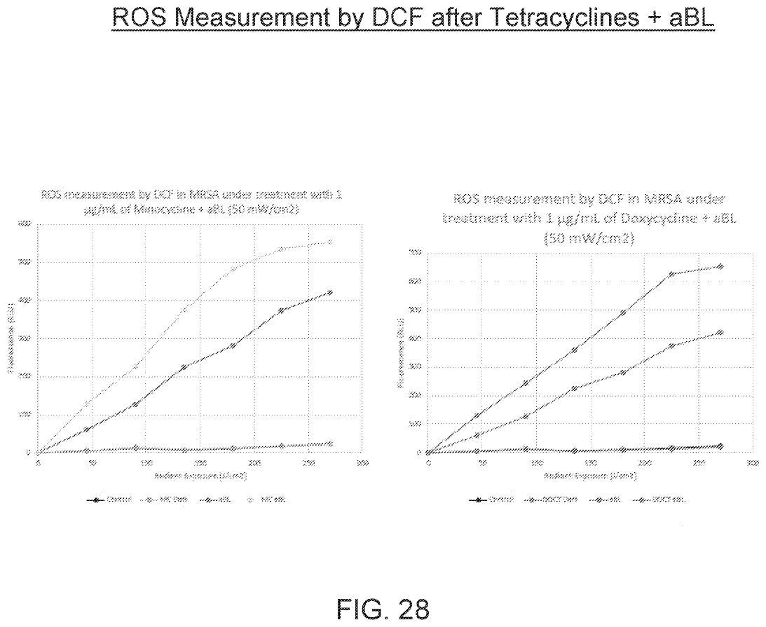

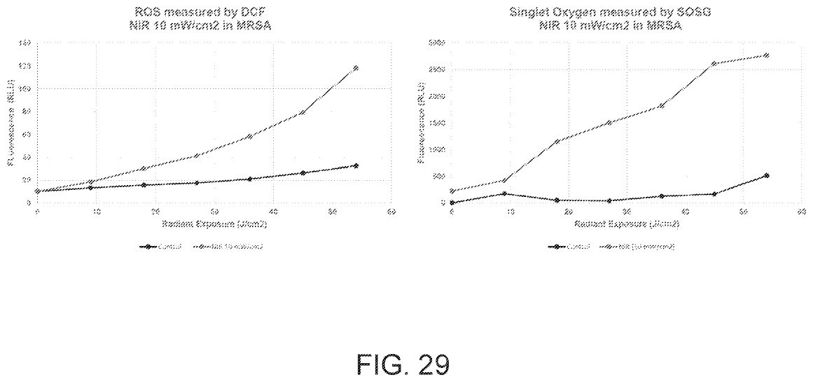

We investigated the possibility that minocycline and doxycycline might be acting as a photosensitizer. To do so, the production of ROS by minocycline and doxycycline combined with blue light was measured by the ROS probe DCF in MRSA. From FIG. 28, it can be observed that each of minocycline and doxycycline when combined with blue light at 405 nm produced ROS.

Thus, we have shown that antimicrobial agents (e.g., tetracyclines) can be used in conjunction with antimicrobial blue light to reduce the bacterial bioburden in biofilms. The use of tetracyclines in a wound dressing of the present invention has the potential to reduce the power consumption because when the light is off, there is still a residual antimicrobial effect to suppress bacteria.

### Example 5

Bacteria in biofilms are metabolically much less active (“dormant”, “persisters”) than planktonic bacteria, rendering them more resistant to antibiotics by 100-1,000-fold, and as we have seen, more resistant to oxidative killing by blue light. Near infrared (NIR-700 nanometers to 2000 nanometers) radiation of low power density can stimulate bacterial growth and energy metabolism. We demonstrated in this Example 5 that bacterial persisters/dormant bacteria in biofilms can be “turned on” by near infrared (NIR) of extremely low dose to become metabolically more active, generating intracellular ROS that can stimulate bacteria in biofilms (“dormant bacteria”, “bacterial persisters”).

We investigated the possibility that NIR radiation at 850 nanometers might be acting as a photosensitizer. To do so, the production of ROS by NIR at 850 nm at 10 mW/cm² was measured by the ROS probes DCF and SOSG in MRSA biofilms. From FIG. 29, it can be observed that NIR at 850 nm produced ROS.

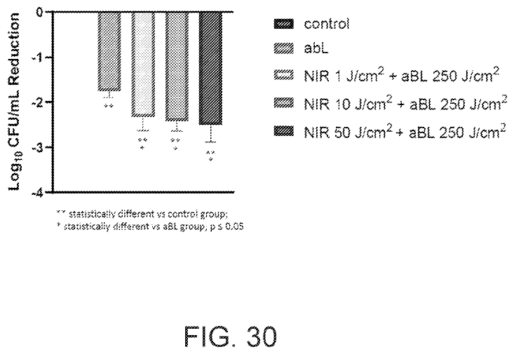

A bacterial suspension was grown in a shaking incubator overnight in BHI broth. Cells were collected by centrifugation at 4,000 rpm for 5 minutes and suspended in PBS at a density of 106 CFU/mL. Then, the biofilms were grown for 48 hours in 96-well microtiter plates with a renewal of the media every 24 hours. After that, the biofilms were: (1) maintained without irradiation (“control”); (2) irradiated with aBL (405 nm) at a radiant exposure 250 J/cm²; (3) irradiated with NIR at 850 nm to a radiant exposure of 1 J/cm² and then irradiated with aBL (405 nm) to a radiant exposure 250 J/cm²; (4) irradiated with NIR at 850 nm to a radiant exposure of 10 J/cm² and then irradiated with aBL (405 nm) to a radiant exposure 250 J/cm²; and (5) irradiated with NIR at 800 nm to a radiant exposure of 50 J/cm² and then irradiated with aBL (405 nm) to a radiant exposure 250 J/cm². After the light exposure, the biofilms were carefully washed two times with PBS and 200 μL of PBS was added to each well. Then, the bacterial biofilms in each well were harvested by scratching with a sterile pipette tip and three wells of each group were pooled together in a 1.5 mL microcentrifuge tube. The total volume obtained, 600 μL (3 wells), was sonicated using a Branson 2510 Water Bath Sonicator (Marshall Scientific, LLC) for 5 minutes and the CFU/mL was determined by 10-fold serial dilutions in PBS on BHI agar plates. CFU/mL was counted after overnight incubation at 37° C. We found an enhancement in antimicrobial effects in MRSA biofilm when aBL was combined with near infrared (NIR) radiation (see FIG. 30).

In Example 5, we have shown that bacterial persisters/dormant bacteria in biofilms can be “turned on” by near infrared (NIR) of extremely low dose to become metabolically more active, rendering the bacteria more susceptible to both antimicrobial blue light and antimicrobial agents.

Thus, the present invention provides methods and devices for improving healing behavior of a biological wound, kits for improving healing behavior of a biological wound, and methods for killing or inhibiting growth of microbes in a biofilm such as bacteria in a biofilm on or adjacent a biological wound. We have developed a Photonic Antimicrobial Wound Surface Dressing (PAWS-Dressing) surface wound covering which delivers antimicrobial blue light. We have shown that antimicrobial adjuvants (e.g., menadione, NIR) and/or antimicrobial agents (e.g., tetracyclines) can be used in conjunction with antimicrobial blue light to reduce the bacterial bioburden in biofilms. Using methods and devices of the invention, one can develop a suite of wound dressings capable of addressing different clinical needs (e.g., “dry” ischemic ulcers, bleeding wounds, deep tissue infections, etc.) employing different wavelengths and configurations.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A device for improving infection healing behavior of a biological wound, the device comprising:
a matrix;
a light-emitting layer comprising a plurality of light sources;
a controller in electrical communication with the light sources, the controller being configured to execute a program stored in the controller to activate the light sources to irradiate the wound or a region adjacent the wound with light for a period of time when the device is placed over the wound; and
an antimicrobial adjuvant present as part of the device in an amount effective to synergistically potentiate antimicrobial activity of the light with respect to microbes on or adjacent the wound.

2. A device for improving infection healing behavior of a biological wound, the device comprising:
a matrix dimensioned to cover the wound, the matrix having a surface including a channel, the surface facing the wound when the device is placed over the wound;
a light-emitting layer comprising a plurality of light sources;
a controller in electrical communication with the light sources, the controller being configured to execute a program stored in the controller to activate the light sources to irradiate the wound or a region adjacent the wound with light for a period of time when the device is placed over the wound; and
a source of irrigation fluid in fluid communication with an inlet of the channel.

3-11. (canceled)

12. The device of claim 1 wherein:
the plurality of light sources comprise an array of light emitting diodes, and
the light-emitting layer comprises at least one lens for focusing or dispersing light from at least a portion of the light emitting diodes.

13. (canceled)

14. (canceled)

15. The device of claim 1 wherein:
the antimicrobial adjuvant comprises a substituted naphthalene.

16. The device of claim 1 wherein:
the antimicrobial adjuvant comprises an oxygen substituted naphthalene.

17. The device of claim 1 wherein:
the antimicrobial adjuvant comprises 2-methyl-1,4-naphthoquinone.

18-26. (canceled)

27. The device of claim 1 further comprising:
an oxygen sensor for sensing oxygen on or adjacent the wound, the oxygen sensor being in electrical communication with the controller.

28. (canceled)

29. (canceled)

30. The device of claim 1 wherein the matrix has a surface including a channel, the surface facing the wound when the device is placed over the wound.

31. The device of claim 30 further comprising:
a source of irrigation fluid in fluid communication with an inlet of the channel.

32. The device of claim 31 wherein:
the irrigation fluid comprises an additive selected from the group consisting of cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, pharmaceuticals, photosensitizers, essential oils, therapeutics, and mixtures thereof.

33. (canceled)

34. The device of claim 31 wherein:
the irrigation fluid comprises oxygen.

35. The device of claim 30 wherein:
the matrix has a surface including a plurality of channels, the surface facing the wound when the device is placed over the wound.

36. The device of claim 35 further comprising:
a source of irrigation fluid in fluid communication with an inlet of the channels.

37. (canceled)

38. (canceled)

39. The device of claim 1 further comprising:
a coolant fluid in contact with the matrix or the light-emitting layer.

40. The device of claim 39 wherein:
the matrix includes a cavity for receiving the coolant fluid.

41. The device of claim 1 further comprising:
a pump for recirculating the coolant fluid, the pump being in fluid communication with the cavity, the pump being in electrical communication with the controller.

42. The device of claim 1 further comprising:
a container including a coolant fluid, wherein the container is in contact with the matrix or the light-emitting layer.

43. The device of claim 39 wherein:
the coolant fluid is selected from biocompatible fluids.

44. The device of claim 43 wherein:
the coolant fluid is a dielectric fluid.

45-74. (canceled)

75. A kit for improving infection healing behavior of a biological wound, the kit comprising:
a matrix dimensioned to cover the wound;
a light-emitting layer comprising a plurality of light sources, the light-emitting layer being configured to irradiate the wound or a region adjacent the wound with light at a range of wavelengths;
a controller configured to be placed in electrical communication with the light sources of the light-emitting layer, the controller being configured to execute a program stored in the controller to activate the light sources of each light-emitting layer to irradiate the wound or the region adjacent the wound when the matrix is placed over the wound; and
an antimicrobial adjuvant for synergistically potentiating antimicrobial activity of the light with respect to microbes on or adjacent the wound.

76-130. (canceled)
